SEQUENCE LISTING (126 sequences)

SEQ ID NO: 1 - protein, 494 aa, unknown
  Lysk endolysin
AKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

SEQ ID NO: 2 - protein, 488 aa, unknown
  PlySK1249 endolysin
GKHLVICGHGQGRTTYDPGAVNAKLGITEAGKVRELAKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFETKKEDIAKGLVSAITGVEVKTIVPSTPSSTVGSSGTPSKPIYLVGDSLRVLPHATHYQTGQKIANWVKGRTYKILQEKNVHQSNSLRAYLLDGIKSWVLEQDVEGTTKGHSEQTYQAQKGDTYYGIARKFGLTVDALLAVNGLKKTDILRVGQTLKVNAASRITTAIPTSVASRVVASALSKVGQKVTVPSNPYGGQCVALVDKIVQELTDKNMSYTNAIDCLKKAKSNGFQVIYDAWGVNPKAGDFYVIETDGLVYGHIGVCVTDSDGKSIDGVEQNIDGYSDHNKNGINDQLEIGGGGITRRVKRQWMADGSLYDSTGTVKLGKVVGWFRIS

SEQ ID NO: 3 - protein, 467 aa, unknown
  lambdaSa2lys
EINTEIAIAWMSARQGKVSYSMDYRDGPNSYDCSSSVYYALRSAGASSAGWAVNTEYMHDWLIKNGYELIAENVDWNAVRGDIAIWGMRGHSSGAGGHVVMFIDPENIIHCNWANNGITVNNYNQTAAASGWMYCYVYRLKSGASTQGKSLDTLVKETLAGNYGNGEARKAVLGNQYEAVMSVINGKTTTNQKTVDQLVQEVIAGKHGNGEARKKSLGSQYDAVQKRVTELLKKQPSEPFKAQEVNKPTETKTSQTELTGQATATKEEGDLSFNGTILKKAVLDKILGNCKKHDILPSYALTILHYEGLWGTSAVGKADNNWGGMTWTGQGNRPSGVTVTQGSARPSNEGGHYMHYASVDDFLTDWFYLLRAGGSYKVSGAKTFSEAIKGMFKVGGAVYDYAASGFDSYIVGASSRLKAIEAENGSLDKFDKATDIGDGSKDKIDITIEGIEVTINGITYELTKKPV

SEQ ID NO: 4 - protein, 340 aa, unknown
  Ply511 endolysin
VKYTVENKIIAGLPKGKLKGANFVIAHETANSKSTIDNEVSYMTRNWKNAFVTHFVGGGGRVVQVANVNYVSWGAGQYANSYSYAQVELCRTSNATTFKKDYEVYCQLLVDLAKKAGIPITLDSGSKTSDKGIKSHKWVADKLGGTTHQDPYAYLSSWGISKAQFASDLAKVSGGGNTGTAPAKPSTPAPKPSTPSTNLDKLGLVDYMNAKKMDSSYSNRDKLAKQYGIANYSGTASQNTTLLSKIKGGAPKPSTPAPKPSTSTAKKIYFPPNKGNWSVYPTNKAPVKANAIGAINPTKFGGLTYTIQKDRGNGVYEIQTDQFGRVQVYGAPSTGAVIKK

SEQ ID NO: 5 - protein, 338 aa, unknown
  Cpl-1 endolysin
VKKNDLFVDVSSHNGYDITGILEQMGTTNTIIKISESTTYLNPCLSAQVEQSNPIGFYHFARFGGDVAEAEREAQFFLDNVPMQVKYLVLDYEDDPSGDAQANTNACLRFMQMIADAGYKPIYYSYKPFTHDNVDYQQILAQFPNSLWIAGYGLNDGTANFEYFPSMDGIRWWQYSSNPFDKNIVLLDDEEDDKPKTAGTWKQDSKGWWFRRNNGSFPYNKWEKIGGVWYYFDSKGYCLTSEWLKDNEKWYYLKDNGAMATGWVLVGSEWYYMDDSGAMVTGWVKYKNNWYYMTNERGNMVSNEFIKSGKGWYFMNTNGELADNPSFTKEPDGLITVA

SEQ ID NO: 6 - protein, 327 aa, unknown
  OBPgp279
KNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKALGIV

SEQ ID NO: 7 - protein, 291 aa, unknown
  EL188 endolysin
NFRTKNGYRDLQALVKELGLYTGQIDGVWGKGTSSSTETLLRGYAEVVGKNTGGIGLPTTSDASGYNVITALQRNLAFLGLYSLTVDGIWGNGTLSGLDKAFEVYKERYRTPTYDIAWSGKVSPAFTAKVKDWCGVHVPNHRAPHWLMACMAFETGQTFSPSIKNAAGSEAYGLIQFMSPAANDLNVPLSVIRSMDQLTQLDLVFKYFEMWMKRGKRYTQLEDFYLTIFHPASVGKKADEVLFLQGSKAYLQNKGFDVDKDGKITLGEISSTLYTTYYKGLLPENRHVISY

SEQ ID NO: 8 - protein, 235 aa, unknown
  PVP-SE1gp146
NAAIAEIQRMLIEGGFSVGKSGADGLYGPATKAALQKCIAQATSGNNKGGTLKLTQAQLDKIFPVGASSGRNAKFLKPLNDLFEKTEINTVNRVAGFLSQIGVESAEFRYVRELGNDAYFDKYDTGPIAERLGNTPQKDGDGAKYKGRGLIQVTGLANYKACGKALGLDLVNHPELLEQPEYAVASAGWYWDTRNINAACDADDIVKITKLVNGGTNHLAERTAYYKKAKSVLTS

SEQ ID NO: 9 - protein, 263 aa, unknown
  Phage003/001_gp110
AILKLGNRGSEVKALQQSLNKIGFSLTADGIFGKATENAVKSVQAGAGLVIDGIAGPKTFYAIRNAGDAHQEHLTEADLVDAARELGVELASMKAVNQVESRGTGFTKTGKIKTLFERHIMYKKVTAKFGQARANALYQLYPTLVNPNSGGYIGGDAELERLQGAIALDEDCAYESASYGLFQIMGFNCQICGYSNAKEMFTDFLTGERAHLLAFVKFIKADANMWKALKNKNWAEFARRYNGPAYAKNQYDTKLAAAYKSFC

SEQ ID NO: 10 - protein, 279 aa, unknown
  Smi01 endolysin
EYDMILKFGSKGDAVATLQKQLAKMGYKGVKDKPLSVDGHFGESTEFAVIQLQRKFGLVADGKVGDKTRQALAGDSVSKFLKDEDYKKAAIRLKVPELVIRVFGAVEGLGVGFLPNGKAKILFERHRMYFYLCQALGKTFANSQVKITPNIVNTLTGGYKGDAAEYTRLSMAINIHKESALMSTSWGQFQIMGENWKDLGYSSVQEFVDQQQLNEGNQLEAFIRFIEWKPGLLEALRKQDWDTVFTLYNGKNYKKLGYQAKFQKEWDHLEPIYREKTAA

SEQ ID NO: 11 - protein, 259 aa, artificial
  KZ144 endolysin
KVLRKGDRGDEVSQLQTLLNLSGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 12 - protein, 244 aa, artificial
  Lysostaphin
AHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 13 - protein, 6 aa, artificial
  synthetic sequence
KRKKRK

SEQ ID NO: 14 - protein, 5 aa, artificial
  synethtic sequence
KRXKR

SEQ ID NO: 15 - protein, 5 aa, artificial
  synthetic sequence
KRSKR

SEQ ID NO: 16 - protein, 5 aa, artificial
  synthetic sequence
KRGSG

SEQ ID NO: 17 - protein, 9 aa, artificial
  synthetic sequence
KRKKRKKRK

SEQ ID NO: 18 - protein, 9 aa, artificial
  synthetic sequence
RRRRRRRRR

SEQ ID NO: 19 - protein, 8 aa, artificial
  synthetic sequence
KKKKKKKK

SEQ ID NO: 20 - protein, 10 aa, artificial
  synthetic sequence
KRKKRKKRKK

SEQ ID NO: 21 - protein, 12 aa, artificial
  synthetic sequence
KRKKRKKRKKRK

SEQ ID NO: 22 - protein, 14 aa, artificial
  synthetic sequence
KRKKRKKRKKRKKR

SEQ ID NO: 23 - protein, 16 aa, artificial
  synthetic sequence
KKKKKKKKKKKKKKKK

SEQ ID NO: 24 - protein, 18 aa, artificial
  synthetic sequence
KRKKRKKRKKRKKRKKRK

SEQ ID NO: 25 - protein, 19 aa, artificial
  synthetic sequence
KRKKRKKRKKRKKRKKRKK

SEQ ID NO: 26 - protein, 19 aa, artificial
  synthetic sequence
RRRRRRRRRRRRRRRRRRR

SEQ ID NO: 27 - protein, 19 aa, artificial
  synthetic sequence
KKKKKKKKKKKKKKKKKKK

SEQ ID NO: 28 - protein, 20 aa, artificial
  synthetic sequence
KRKKRKKRKRSKRKKRKKRK

SEQ ID NO: 29 - protein, 21 aa, artificial
  synthetic sequence
KRKKRKKRKRSKRKKRKKRKK

SEQ ID NO: 30 - protein, 21 aa, artificial
  synthetic sequence
KRKKRKKRKKRKKRKKRKKRK

SEQ ID NO: 31 - protein, 22 aa, artificial
  synthetic sequence
KRKKRKKRKRGSGKRKKRKKRK

SEQ ID NO: 32 - protein, 24 aa, artificial
  synthetic sequence
KRKKRKKRKRGSGSGKRKKRKKRK

SEQ ID NO: 33 - protein, 25 aa, artificial
  synthetic sequence
KRKKRKKRKKRKKRKKRKKRKKRKK

SEQ ID NO: 34 - protein, 31 aa, artificial
  synthetic sequence
KRKKRKKRKRSKRKKRKKRKRSKRKKRKKRK

SEQ ID NO: 35 - protein, 38 aa, artificial
  synthetic sequence
KRKKRKKRKRGSGSGKRKKRKKRKGSGSGKRKKRKKRK

SEQ ID NO: 36 - protein, 39 aa, artificial
  synthetic sequence
KRKKRKKRKKRKKRKKRKKRKKRKKRKKRKKRKKRKKRK

SEQ ID NO: 37 - protein, 42 aa, artificial
  synthetic sequence
KRKKRKKRKRSKRKKRKKRKRSKRKKRKKRKRSKRKKRKKRK

SEQ ID NO: 38 - protein, 37 aa, Homo sapiens
LLGDFFRKSKEKIGKEFKRIVQRIKDFLRNLVPRTES

SEQ ID NO: 39 - protein, 29 aa, unknown
  SMAP-29 sheep
RGLRRLGRKIAHGVKKYGPTVLRIIRIAG

SEQ ID NO: 40 - protein, 13 aa, unknown
  Indolicidine bovine
ILPWKWPWWPWRR

SEQ ID NO: 41 - protein, 18 aa, unknown
  Protegrin Porcine
RGGRLCYCRRRFCVCVGR

SEQ ID NO: 42 - protein, 31 aa, unknown
  Cecropin P1 Mammal (pig)
SWLSKTAKKLENSAKKRISEGIAIAIQGGPR

SEQ ID NO: 43 - protein, 23 aa, unknown
  Magainin frog
GIGKFLHSAKKFGKAFVGEIMNS

SEQ ID NO: 44 - protein, 25 aa, unknown
  Pleurocidin fish
GWGSFFKKAAHVGKHVGKAALTHYL

SEQ ID NO: 45 - protein, 36 aa, Aedes aegypti
GGLKKLGKKLEGAGKRVFNAAEKALPVVAGAKALRK

SEQ ID NO: 46 - protein, 40 aa, Drosophila melanogaster
GWLKKIGKKIERVGQHTRDATIQGLGIPQQAANVAATARG

SEQ ID NO: 47 - protein, 21 aa, unknown
  Buforin II vertebrate
TRSSRAGLQFPVGRVHRLLRK

SEQ ID NO: 48 - protein, 39 aa, unknown
  Sarcotoxin IA Fly
GWLKKIGKKIERVGQHTRDATIQGLGIAQQAANVAATAR

SEQ ID NO: 49 - protein, 17 aa, Apis mellifera
ANRPVYIPPPRPPHPRL

SEQ ID NO: 50 - protein, 24 aa, unknown
  Ascaphine 5 Frog
GIKDWIKGAAKKLIKTVASHIANQ

SEQ ID NO: 51 - protein, 22 aa, unknown
  Nigrocine 2 Frog
GLLSKVLGVGKKVLCGVSGLVC

SEQ ID NO: 52 - protein, 24 aa, unknown
  Pseudin 1 Rana Frog
GLNTLKKVFQGLHEAIKLINNHVQ

SEQ ID NO: 53 - protein, 18 aa, unknown
  Ranalexin Frog
FLGGLIVPAMICAVTKKC

SEQ ID NO: 54 - protein, 26 aa, unknown
  Melittin bee
GIGAVLKVLTTGLPALISWIKRKRQQ

SEQ ID NO: 55 - protein, 25 aa, unknown
  Lycotoxin 1 Spider
IWLTALKFLGKHAAKKLAKQQLSKL

SEQ ID NO: 56 - protein, 19 aa, unknown
  Parasin 1 Fish
KGRGKQGGKVRAKAKTRSS

SEQ ID NO: 57 - protein, 39 aa, unknown
  Buforin I Toad
AGRGKQGGKVRAKAKTRSSRAGLQFPVGRVHRLLRKGNY

SEQ ID NO: 58 - protein, 34 aa, unknown
  Dermaseptin 1 Frog
ALWKTMLKKLGTMALHAGKAALGAAADTISQGTQ

SEQ ID NO: 59 - protein, 12 aa, unknown
  Bactenecin 1 Cow
RLCRIVVIRVCR

SEQ ID NO: 60 - protein, 21 aa, unknown
  Thanatin Insect
GSKKPVPIIYCNRRTGKCQRM

SEQ ID NO: 61 - protein, 19 aa, unknown
  Brevinin 1T Rana frogs
VNPIILGVLPKVCLITKKC

SEQ ID NO: 62 - protein, 26 aa, unknown
  Ranateurin 1 Rana frog
SMLSVLKNLGKVGLGFVACKINIKQC

SEQ ID NO: 63 - protein, 46 aa, unknown
  Esculentin 1 Rana frogs
GIFSKLGRKKIKNLLISGLKNVGKEVGMDVVRTGIKIAGCKIKGEC

SEQ ID NO: 64 - protein, 17 aa, Limulus polyphemus
RWCFRVCYRGICYRKCR

SEQ ID NO: 65 - protein, 25 aa, unknown
  Androctonin Scorpion
RSVCRQIKICRRRGGCYYKCTNRPY

SEQ ID NO: 66 - protein, 30 aa, Homo sapiens
DCYCRIPACIAGERRYGTCIYQGRLWAFCC

SEQ ID NO: 67 - protein, 38 aa, unknown
  beta-defensin cow
NPVSCVRNKGICVPIRCPGSMKQIGTCVGRAVKCCRKK

SEQ ID NO: 68 - protein, 18 aa, unknown
  theta-defensin monkey
GFCRCLCRRGVCRCICTR

SEQ ID NO: 69 - protein, 40 aa, unknown
  defensin (sapecin A) insect
ATCDLLSGTGINHSACAAHCLLRGNRGGYCNGKAVCVCRN

SEQ ID NO: 70 - protein, 46 aa, unknown
  Thionin (crambin) plant
TTCCPSIVARSNFNVCRIPGTPEAICATYTGCIIIPGATCPGDYAN

SEQ ID NO: 71 - protein, 50 aa, unknown
  defensin from radish
QKLCQRPSGTWSGVCGNNNACKNQCIRLEKARHGSCNYVFPAHCICYFPC

SEQ ID NO: 72 - protein, 44 aa, Drosophila melanogaster
DCLSGRYKGPCAVWDNETCRRVCKEEGRSSGHCSPSLKCWCEGC

SEQ ID NO: 73 - protein, 25 aa, Homo sapiens
DTHFPICIFCCGCCHRSKCGMCCKT

SEQ ID NO: 74 - protein, 44 aa, unknown
  Bac 5 Cow
RFRPPIRRPPIRPPFYPPFRPPIRPPIFPPIRPPFRPPLGRPFP

SEQ ID NO: 75 - protein, 39 aa, unknown
  PR-39 Pig
RRRPRPPYLPRPRPPPFFPPRLPPRIPPGFPPRFPPRFP

SEQ ID NO: 76 - protein, 20 aa, unknown
  Pyrrhocoricin Insect
VDKGSYLPRPTPPRPIYNRN

SEQ ID NO: 77 - protein, 24 aa, Homo sapiens
DSHAKRHHGYKRKFHEKHHSHRGY

SEQ ID NO: 78 - protein, 26 aa, unknown
  Macedocin
GKNGVFKTISHECHLNTWAFLATCCS

SEQ ID NO: 79 - protein, 22 aa, unknown
  Macedocin (Trunc)
GKNGVFKTISHECHLNTWAFLA

SEQ ID NO: 80 - protein, 28 aa, unknown
  D16
ACKLKSLLKTLSKAKKKKLKTLLKALSK

SEQ ID NO: 81 - protein, 17 aa, unknown
  CPF-C1
GFGSLLGKALRLGANVL

SEQ ID NO: 82 - protein, 34 aa, unknown
  TL-ColM
ETLTVHAPSPSTNLPSYGNGAFSLSAPHVPGAGP

SEQ ID NO: 83 - protein, 26 aa, unknown
  TM-174E
LISKGWPYLLVVVLGATIYFWGNSNG

SEQ ID NO: 84 - protein, 23 aa, unknown
  MSI-594
GIGKFLKKAKKGIGAVLKVLTTG

SEQ ID NO: 85 - protein, 19 aa, unknown
  ECP19
RPPQFTRAQWFAIQHISLN

SEQ ID NO: 86 - protein, 45 aa, unknown
  ECP45
RPPQFTRAQWFAIQHISLNPPRCTIAMRAINNYRWRCKNQNTFLR

SEQ ID NO: 87 - protein, 50 aa, unknown
  ColicinE3_1-51 (S37F)
SGGDGRGHNTGAHSTSGNINGGPTGLGVGGGASDGFGWSSENNPWGGGSG

SEQ ID NO: 88 - protein, 68 aa, unknown
  ColicinE3_1-69 (S37F)
SGGDGRGHNTGAHSTSGNINGGPTGLGVGGGASDGFGWSSENNPWGGGSGSGIHWGGGSGHGNGGGNG

SEQ ID NO: 89 - protein, 52 aa, unknown
  ColicinD_1-53
SDYEGSGPTEGIDYGHSMVVWPSTGLISGGDVKPGGSSGIAPSMPPGWGDYS

SEQ ID NO: 90 - protein, 34 aa, Limulus polyphemus
GFKLKGMARISCLPNGQWSNFPPKCIRECAMVSS

SEQ ID NO: 91 - protein, 18 aa, artificial
  synthetic sequence
GFFIPAVILPSIAFLIVP

SEQ ID NO: 92 - protein, 5 aa, artificial
  synthetic sequence
FFVAP

SEQ ID NO: 93 - protein, 13 aa, unknown
  alpha4-helix of T4 lysozyme
PNRAKRVITTFRT

SEQ ID NO: 94 - protein, 27 aa, artificial
  synthetic sequence
KRWVKRVKRVKRWVKRVVRVVKRWVKR

SEQ ID NO: 95 - protein, 25 aa, artificial
  synthetic sequence
GKPGWLIKKALVFKKLIRRPLKRLA

SEQ ID NO: 96 - protein, 253 aa, artificial
  Lysostaphin-PK
AHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKKRKKRKKRK

SEQ ID NO: 97 - protein, 283 aa, artificial
  SarcotoxinIA-Lysostaphin
GWLKKIGKKIERVGQHTRDATIQGLGIAQQAANVAATARAHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 98 - protein, 517 aa, artificial
  LysK-Magainin
AKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKGIGKFLHSAKKFGKAFVGEIMNS

SEQ ID NO: 99 - protein, 514 aa, artificial
  LysK-Ranalexin
AKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKFLGGLIKIVPAMICAVTKKC

SEQ ID NO: 100 - protein, 497 aa, artificial
  PlySK1249-PK
GKHLVICGHGQGRTTYDPGAVNAKLGITEAGKVRELAKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFETKKEDIAKGLVSAITGVEVKTIVPSTPSSTVGSSGTPSKPIYLVGDSLRVLPHATHYQTGQKIANWVKGRTYKILQEKNVHQSNSLRAYLLDGIKSWVLEQDVEGTTKGHSEQTYQAQKGDTYYGIARKFGLTVDALLAVNGLKKTDILRVGQTLKVNAASRITTAIPTSVASRVVASALSKVGQKVTVPSNPYGGQCVALVDKIVQELTDKNMSYTNAIDCLKKAKSNGFQVIYDAWGVNPKAGDFYVIETDGLVYGHIGVCVTDSDGKSIDGVEQNIDGYSDHNKNGINDQLEIGGGGITRRVKRQWMADGSLYDSTGTVKLGKVVGWFRISKRKKRKKRK

SEQ ID NO: 101 - protein, 509 aa, artificial
  BuforinII-PlySK1249
TRSSRAGLQFPVGRVHRLLRKGKHLVICGHGQGRTTYDPGAVNAKLGITEAGKVRELAKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFETKKEDIAKGLVSAITGVEVKTIVPSTPSSTVGSSGTPSKPIYLVGDSLRVLPHATHYQTGQKIANWVKGRTYKILQEKNVHQSNSLRAYLLDGIKSWVLEQDVEGTTKGHSEQTYQAQKGDTYYGIARKFGLTVDALLAVNGLKKTDILRVGQTLKVNAASRITTAIPTSVASRVVASALSKVGQKVTVPSNPYGGQCVALVDKIVQELTDKNMSYTNAIDCLKKAKSNGFQVIYDAWGVNPKAGDFYVIETDGLVYGHIGVCVTDSDGKSIDGVEQNIDGYSDHNKNGINDQLEIGGGGITRRVKRQWMADGSLYDSTGTVKLGKVVGWFRIS

SEQ ID NO: 102 - protein, 507 aa, artificial
  lambdaSa2lys-Cecropin A (D.me)
EINTEIAIAWMSARQGKVSYSMDYRDGPNSYDCSSSVYYALRSAGASSAGWAVNTEYMHDWLIKNGYELIAENVDWNAVRGDIAIWGMRGHSSGAGGHVVMFIDPENIIHCNWANNGITVNNYNQTAAASGWMYCYVYRLKSGASTQGKSLDTLVKETLAGNYGNGEARKAVLGNQYEAVMSVINGKTTTNQKTVDQLVQEVIAGKHGNGEARKKSLGSQYDAVQKRVTELLKKQPSEPFKAQEVNKPTETKTSQTELTGQATATKEEGDLSFNGTILKKAVLDKILGNCKKHDILPSYALTILHYEGLWGTSAVGKADNNWGGMTWTGQGNRPSGVTVTQGSARPSNEGGHYMHYASVDDFLTDWFYLLRAGGSYKVSGAKTFSEAIKGMFKVGGAVYDYAASGFDSYIVGASSRLKAIEAENGSLDKFDKATDIGDGSKDKIDITIEGIEVTINGITYELTKKPVGWLKKIGKKIERVGQHTRDATIQGLGIPQQAANVAATARG

SEQ ID NO: 103 - protein, 490 aa, artificial
  Magainin-lambdaSa2lys
GIGKFLHSAKKFGKAFVGEIMNSEINTEIAIAWMSARQGKVSYSMDYRDGPNSYDCSSSVYYALRSAGASSAGWAVNTEYMHDWLIKNGYELIAENVDWNAVRGDIAIWGMRGHSSGAGGHVVMFIDPENIIHCNWANNGITVNNYNQTAAASGWMYCYVYRLKSGASTQGKSLDTLVKETLAGNYGNGEARKAVLGNQYEAVMSVINGKTTTNQKTVDQLVQEVIAGKHGNGEARKKSLGSQYDAVQKRVTELLKKQPSEPFKAQEVNKPTETKTSQTELTGQATATKEEGDLSFNGTILKKAVLDKILGNCKKHDILPSYALTILHYEGLWGTSAVGKADNNWGGMTWTGQGNRPSGVTVTQGSARPSNEGGHYMHYASVDDFLTDWFYLLRAGGSYKVSGAKTFSEAIKGMFKVGGAVYDYAASGFDSYIVGASSRLKAIEAENGSLDKFDKATDIGDGSKDKIDITIEGIEVTINGITYELTKKPV

SEQ ID NO: 104 - protein, 349 aa, artificial
  Ply511-PK
VKYTVENKIIAGLPKGKLKGANFVIAHETANSKSTIDNEVSYMTRNWKNAFVTHFVGGGGRVVQVANVNYVSWGAGQYANSYSYAQVELCRTSNATTFKKDYEVYCQLLVDLAKKAGIPITLDSGSKTSDKGIKSHKWVADKLGGTTHQDPYAYLSSWGISKAQFASDLAKVSGGGNTGTAPAKPSTPAPKPSTPSTNLDKLGLVDYMNAKKMDSSYSNRDKLAKQYGIANYSGTASQNTTLLSKIKGGAPKPSTPAPKPSTSTAKKIYFPPNKGNWSVYPTNKAPVKANAIGAINPTKFGGLTYTIQKDRGNGVYEIQTDQFGRVQVYGAPSTGAVIKKKRKKRKKRK

SEQ ID NO: 105 - protein, 351 aa, artificial
  Cpl-1-Indolicidin
VKKNDLFVDVSSHNGYDITGILEQMGTTNTIIKISESTTYLNPCLSAQVEQSNPIGFYHFARFGGDVAEAEREAQFFLDNVPMQVKYLVLDYEDDPSGDAQANTNACLRFMQMIADAGYKPIYYSYKPFTHDNVDYQQILAQFPNSLWIAGYGLNDGTANFEYFPSMDGIRWWQYSSNPFDKNIVLLDDEEDDKPKTAGTWKQDSKGWWFRRNNGSFPYNKWEKIGGVWYYFDSKGYCLTSEWLKDNEKWYYLKDNGAMATGWVLVGSEWYYMDDSGAMVTGWVKYKNNWYYMTNERGNMVSNEFIKSGKGWYFMNTNGELADNPSFTKEPDGLITVAILPWKWPWWPWRR

SEQ ID NO: 106 - protein, 361 aa, artificial
  Magainin-Cpl-1
GIGKFLHSAKKFGKAFVGEIMNSVKKNDLFVDVSSHNGYDITGILEQMGTTNTIIKISESTTYLNPCLSAQVEQSNPIGFYHFARFGGDVAEAEREAQFFLDNVPMQVKYLVLDYEDDPSGDAQANTNACLRFMQMIADAGYKPIYYSYKPFTHDNVDYQQILAQFPNSLWIAGYGLNDGTANFEYFPSMDGIRWWQYSSNPFDKNIVLLDDEEDDKPKTAGTWKQDSKGWWFRRNNGSFPYNKWEKIGGVWYYFDSKGYCLTSEWLKDNEKWYYLKDNGAMATGWVLVGSEWYYMDDSGAMVTGWVKYKNNWYYMTNERGNMVSNEFIKSGKGWYFMNTNGELADNPSFTKEPDGLITVA

SEQ ID NO: 107 - protein, 336 aa, artificial
  PK-OBPgp279
KRKKRKKRKKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKALGIV

SEQ ID NO: 108 - protein, 356 aa, artificial
  SMAP-29-OBPgp279
RGLRRLGRKIAHGVKKYGPTVLRIIRIAGKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKALGIV

SEQ ID NO: 109 - protein, 320 aa, artificial
  SMAP-29-EL188
RGLRRLGRKIAHGVKKYGPTVLRIIRIAGNFRTKNGYRDLQALVKELGLYTGQIDGVWGKGTSSSTETLLRGYAEVVGKNTGGIGLPTTSDASGYNVITALQRNLAFLGLYSLTVDGIWGNGTLSGLDKAFEVYKERYRTPTYDIAWSGKVSPAFTAKVKDWCGVHVPNHRAPHWLMACMAFETGQTFSPSIKNAAGSEAYGLIQFMSPAANDLNVPLSVIRSMDQLTQLDLVFKYFEMWMKRGKRYTQLEDFYLTIFHPASVGKKADEVLFLQGSKAYLQNKGFDVDKDGKITLGEISSTLYTTYYKGLLPENRHVISY

SEQ ID NO: 110 - protein, 300 aa, artificial
  PK-EL188
KRKKRKKRKNFRTKNGYRDLQALVKELGLYTGQIDGVWGKGTSSSTETLLRGYAEVVGKNTGGIGLPTTSDASGYNVITALQRNLAFLGLYSLTVDGIWGNGTLSGLDKAFEVYKERYRTPTYDIAWSGKVSPAFTAKVKDWCGVHVPNHRAPHWLMACMAFETGQTFSPSIKNAAGSEAYGLIQFMSPAANDLNVPLSVIRSMDQLTQLDLVFKYFEMWMKRGKRYTQLEDFYLTIFHPASVGKKADEVLFLQGSKAYLQNKGFDVDKDGKITLGEISSTLYTTYYKGLLPENRHVISY

SEQ ID NO: 111 - protein, 256 aa, artificial
  PVP-SE1gp146-Buforin II
NAAIAEIQRMLIEGGFSVGKSGADGLYGPATKAALQKCIAQATSGNNKGGTLKLTQAQLDKIFPVGASSGRNAKFLKPLNDLFEKTEINTVNRVAGFLSQIGVESAEFRYVRELGNDAYFDKYDTGPIAERLGNTPQKDGDGAKYKGRGLIQVTGLANYKACGKALGLDLVNHPELLEQPEYAVASAGWYWDTRNINAACDADDIVKITKLVNGGTNHLAERTAYYKKAKSVLTSTRSSRAGLQFPVGRVHRLLRK

SEQ ID NO: 112 - protein, 248 aa, artificial
  PVP-SE1gp146-Indolicidin
NAAIAEIQRMLIEGGFSVGKSGADGLYGPATKAALQKCIAQATSGNNKGGTLKLTQAQLDKIFPVGASSGRNAKFLKPLNDLFEKTEINTVNRVAGFLSQIGVESAEFRYVRELGNDAYFDKYDTGPIAERLGNTPQKDGDGAKYKGRGLIQVTGLANYKACGKALGLDLVNHPELLEQPEYAVASAGWYWDTRNINAACDADDIVKITKLVNGGTNHLAERTAYYKKAKSVLTSILPWKWPWWPWRR

SEQ ID NO: 113 - protein, 284 aa, artificial
  Phage003/001_gp110 - Nigrocin 2
AILKLGNRGSEVKALQQSLNKIGFSLTADGIFGKATENAVKSVQAGAGLVIDGIAGPKTFYAIRNAGDAHQEHLTEADLVDAARELGVELASMKAVNQVESRGTGFTKTGKIKTLFERHIMYKKVTAKFGQARANALYQLYPTLVNPNSGGYIGGDAELERLQGAIALDEDCAYESASYGLFQIMGFNCQICGYSNAKEMFTDFLTGERAHLLAFVKFIKADANMWKALKNKNWAEFARRYNGPAYAKNQYDTKLAAAYKSFCGLLSKVLGVGKKVLCGVSGLC

SEQ ID NO: 114 - protein, 288 aa, artificial
  Pleuricidin - Phage003/001_gp110
GWGSFFKKAAHVGKHVGKAALTHYLAILKLGNRGSEVKALQQSLNKIGFSLTADGIFGKATENAVKSVQAGAGLVIDGIAGPKTFYAIRNAGDAHQEHLTEADLVDAARELGVELASMKAVNQVESRGTGFTKTGKIKTLFERHIMYKKVTAKFGQARANALYQLYPTLVNPNSGGYIGGDAELERLQGAIALDEDCAYESASYGLFQIMGFNCQICGYSNAKEMFTDFLTGERAHLLAFVKFIKADANMWKALKNKNWAEFARRYNGPAYAKNQYDTKLAAAYKSFC

SEQ ID NO: 115 - protein, 315 aa, artificial
  Cecropin A (A.aegypti)-Smi01
GGLKKLGKKLEGAGKRVFNAAEKALPVVAGAKALRKEYDMILKFGSKGDAVATLQKQLAKMGYKGVKDKPLSVDGHFGESTEFAVIQLQRKFGLVADGKVGDKTRQALAGDSVSKFLKDEDYKKAAIRLKVPELVIRVFGAVEGLGVGFLPNGKAKILFERHRMYFYLCQALGKTFANSQVKITPNIVNTLTGGYKGDAAEYTRLSMAINIHKESALMSTSWGQFQIMGENWKDLGYSSVQEFVDQQQLNEGNQLEAFIRFIEWKPGLLEALRKQDWDTVFTLYNGKNYKKLGYQAKFQKEWDHLEPIYREKTAA

SEQ ID NO: 116 - protein, 318 aa, artificial
  SarcotoxinIA-Smi01
GWLKKIGKKIERVGQHTRDATIQGLGIAQQAANVAATAREYDMILKFGSKGDAVATLQKQLAKMGYKGVKDKPLSVDGHFGESTEFAVIQLQRKFGLVADGKVGDKTRQALAGDSVSKFLKDEDYKKAAIRLKVPELVIRVFGAVEGLGVGFLPNGKAKILFERHRMYFYLCQALGKTFANSQVKITPNIVNTLTGGYKGDAAEYTRLSMAINIHKESALMSTSWGQFQIMGENWKDLGYSSVQEFVDQQQLNEGNQLEAFIRFIEWKPGLLEALRKQDWDTVFTLYNGKNYKKLGYQAKFQKEWDHLEPIYREKTAA

SEQ ID NO: 117 - protein, 295 aa, artificial
  Cecropin A (A.aegypti)-KZ144
GGLKKLGKKLEGAGKRVFNAAEKALPVVAGAKALRKKVLRKGDRGDEVSQLQTLLNLSGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 118 - protein, 298 aa, artificial
  SarcotoxinIA-KZ144
GWLKKIGKKIERVGQHTRDATIQGLGIAQQAANVAATARKVLRKGDRGDEVSQLQTLLNLSGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 119 - protein, 288 aa, artificial
  SMAP29-KZ144
RGLRRLGRKIAHGVKKYGPTVLRIIRIAGKVLRKGDRGDEVSQLQTLLNLSGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 120 - protein, 4 aa, artificial
  linker
GAGA

SEQ ID NO: 121 - protein, 8 aa, artificial
  linker
GAGAGAGA

SEQ ID NO: 122 - protein, 12 aa, artificial
  linker
GAGAGAGAGAGA

SEQ ID NO: 123 - protein, 299 aa, Artificial Sequence
  SMAP29-KZ144 his
MRGLRRLGRKIAHGVKKYGPTVLRIIRIAGGSKVLRKGDRGDEVSQLQTLLNLSGYDVGKPDGIFGNNTFNQVVKFQKDNSLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRKLEHHHHHH

SEQ ID NO: 124 - protein, 300 aa, artificial
  SarcotoxinIA-KZ144his
MKFFRKLKKSVKKRAKEFFKKPRVIGVSIPFGSKVLRKGDRGDEVCQLQTLLNLCGYDVGKPDGIFGNNTFNQVVKFQKDNCLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRKLEHHHHHH

SEQ ID NO: 125 - protein, 374 aa, Artificial sequence
  CecropinA(A. aegyptii)-OBPlyshis
MGGLKKLGKKLEGAGKRVFNAAEKALPVVAGAKALRKGSKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKALGIVLEHHHHHH

SEQ ID NO: 126 - protein, 299 aa, artificial sequence; identical to SEQ ID NO: 123
  mutated KZ144 with C14S, C23S and C50Splus SMAP-29 and HisTag

(19) United States
(12) Patent Application Publication
(10) Pub. No.: US 2017/0106058 A1
(43) Pub. Date: Apr. 20, 2017

(54) PHARMACEUTICAL COMPOSITION AGAINST CHRONIC BACTERIAL INFECTIONS

(71) Applicant: Lysando AG, Triesenberg (LI)

(72) Inventors: Stefan MILLER, Regensburg (DE); Yves BRIERS, Nederokkerzeel (BE); Rob LAVIGNE, Ekeren (BE)

(21) Appl. No.: 15/301,500

(22) PCT Filed: Apr. 8, 2015
(86) PCT No.: PCT/EP2015/057625

§ 371 Date: Oct. 3, 2016

(30) Foreign Application Priority Data
Apr. 8, 2014 (EP) 14163927.8

(51) Int. Cl. A61K 38/48; C07K 14/435; A61K 47/18; A61K 38/47; A61K 38/50

(52) U.S. Cl. CPC A61K 38/48; A61K 38/47; A61K 38/50; A61K 38/4886; C12Y 302/01017; C12Y 305/01028; A61K 47/183; C07K 14/43577; C07K 2319/55

(57) ABSTRACT

The present invention relates to a polypeptide comprising a peptidoglycan hydrolase for use as a medicament for the treatment of chronic bacterial infections. The present invention further relates to a polypeptide comprising a peptidoglycan hydrolase comprising an endopeptidase, N-acetyl-muramoyl-L-alanine-amidase, N-acetyl-muramidase, N-acetyl-glucosaminidase and/or lytic transglycosylase.

The present invention relates to a polypeptide comprising a peptidoglycan hydrolase for use as a medicament for the treatment of chronic bacterial infections. The present invention further relates to a polypeptide comprising a peptidoglycan hydrolase comprising an endopeptidase, N-acetyl-muramoyl-L-alanine-amidase, N-acetyl-muramidase, N-acetyl-glucosaminidase and/or lytic transglycosylase.

One of the challenges in bacterial infectious diseases is the problem of chronic infections, which require long durations of antibiotic treatment and often reoccur. Chronic bacterial infections are typically caused by bacteria “persisters”, i.e. a small subpopulation of bacteria that survive an antibiotic treatment by essentially shutting down metabolism, even as their counterparts, who are in a normal metabolic state, are killed off. As a result, the patient initially appears to be fully recovered, but over the course of weeks or months, the persisters start returning to life, often stronger and more aggressive than ever before, and the illness is back. Said bacterial persistence is a major obstacle to the successful treatment of infectious bacterial diseases. It can stretch illnesses out over months, cause bacterial infections to spread to kidneys and other organs, and raise treatment costs extremely. Unlike antibiotic resistant bacteria, whose ability to withstand drug treatments is based on genetic differences, persisters are genetically identical to the other members of their bacterial community. Persisters are often called antibiotics tolerant bacterial strains. Given its significant negative impact, bacterial persistence has become a growing threat. Moreover, up to date, no treatment that directly targets persisters is known. Bacterial persistence has been verified in a number of species, such as Escherichia coli, Pseudomonas aeruginosa, Staphylococcus spp., Acinetobacter baumannii, Mycobacterium spp., Listeria monocytogenes, Salmonella. It is assumed that all bacterial pathogens can establish life-long chronic infections.

Thus, there is a need for antibacterial agents that treat chronic bacterial infections.

This object is solved by the subject matter defined in the claims.

The following figure serves to illustrate the invention.

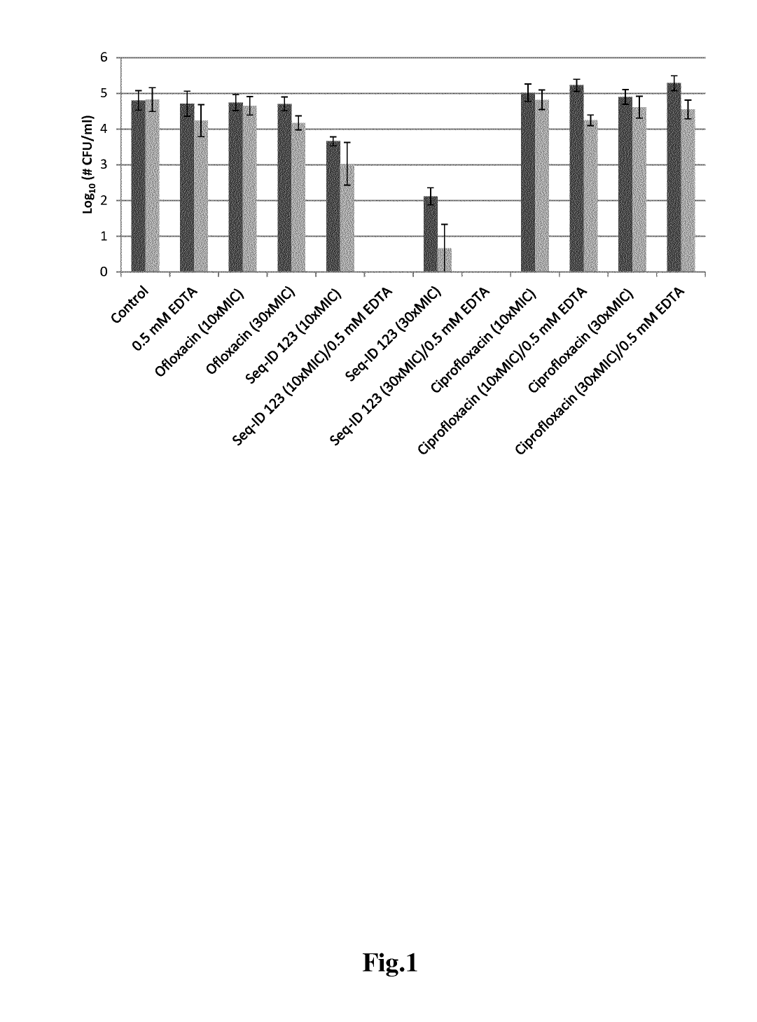

FIG. 1 shows in a schematic diagram the bactericidal effect of a polypeptide according to SEQ ID NO: 123 (10× and 30× MIC) on isolated persister fractions of P. aeruginosa PA14 (light grey) and PA1255 (dark grey) is compared to ciprofloxacin (10× MIC and 30× MIC), both in the absence and presence of 0.5 mM EDTA. Controls include 0.5 mM EDTA and ofloxacin (10× MIC and 30× MIC). Mean values (±SEMs) are shown for at least three independent replicates.

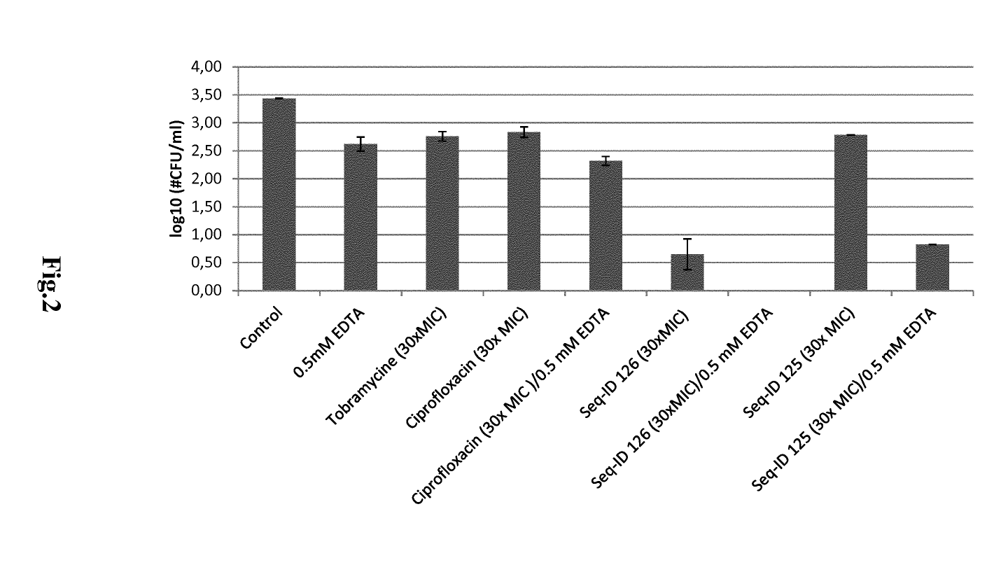

FIG. 2 shows A. baumannii RUH134 persister killing by the polypeptide according to SEQ ID NO: 126 and the polypeptide according to SEQ ID NO: 125. The bactericidal effects of the polypeptide according to SEQ ID NO: 126 (30× MIC) and the polypeptide according to SEQ ID NO: 125 (30× MIC) on isolated persister fractions are compared to those of ciprofloxacin (30× MIC), in both the absence and the presence of 0.5 mM EDTA. Additional controls include 0.5 mM EDTA and tobramycin (30× MIC). Mean values (±standard error of the mean [SEM]) are shown for at least three independent replicates (except for the polypeptide according to SEQ ID NO: 125). The control shows the number of persisters before treatment.

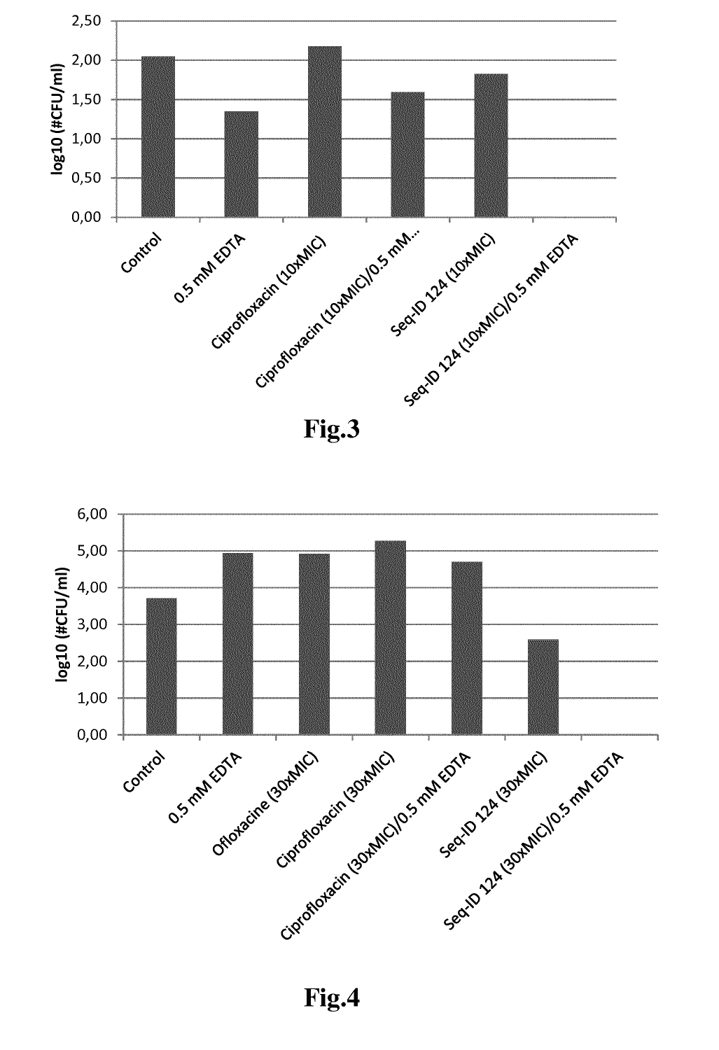

FIG. 3 shows E. coli UTI-89 persister killing by the polypeptide according to SEQ ID NO: 124. The bactericidal effects of the polypeptide according to SEQ ID NO: 124 (10× MIC) on isolated persister fractions are compared to those of ciprofloxacin (10× MIC), in both the absence and the presence of 0.5 mM EDTA. Additional control is 0.5 mM EDTA.

FIG. 4 shows P. aeruginosa PA14 persister killing by the polypeptide according to SEQ ID NO: 124. The bactericidal effects of the polypeptide according to SEQ ID NO: 124 (30×MIC) on isolated persister fractions are compared to those of ciprofloxacin (30× MIC), in both the absence and the presence of 0.5 mM EDTA. Additional controls include 0.5 mM EDTA and ofloxacin

The term “polypeptide” as used herein refers in particular to a polymer of amino acid residues linked by peptide bonds in a specific sequence. The amino acid residues of a polypeptide may be modified by e.g. covalent attachments of various groups such as carbohydrates and phosphate. Other substances may be more loosely associated with the polypeptide, such as heme or lipid, giving rise to conjugated polypeptides which are also comprised by the term “polypeptide” as used herein. The term as used herein is intended to encompass also proteins. Thus, the term “polypeptide” also encompasses for example complexes of two or more amino acid polymer chains. The term “polypeptide” does encompass embodiments of polypeptides which exhibit optionally modifications typically used in the art, e.g. biotinylation, acetylation, pegylation, chemical changes of the amino-, SH- or carboxyl-groups (e.g. protecting groups) etc. As will become apparent from the description below, the polypeptide of to the present invention may also be a fusion protein, i.e. linkage of at least two amino acid sequences which do not occur in this combination in nature. The term “polypeptide” as used herein is not limited to a specific length of the amino acid polymer chain, but typically the polypeptide will exhibit a length of more than about 50 amino acids, more than about 100 amino acids or even more than about 150 amino acids. Usually, but not necessarily, a typical polypeptide of the present invention will not exceed about 750 amino acids in length.

The term “cell wall” as used herein refers to all components that form the outer cell enclosure of Gram-negative and Gram-positive bacteria and thus guarantee their integrity. In particular, the term “cell wall” as used herein refers to peptidoglycan, the outer membrane of the Gram-negative and peptidoglycan of Gram-positive bacteria with the lipopolysaccharide, the bacterial cell membrane, but also to additional layers deposited on the peptidoglycan as e.g. capsules, outer protein layers or slimes.

The term “amino acid sequence stretch” as used herein refers to a particular stretch of amino acid sequence in the amino acid sequence of the polypeptide of the invention. Said sequence refers to a sequence of a cationic peptide, a polycationic peptide, an amphiphatic peptide, a hydrophobic peptide, a sushi peptide and/or an antimicrobial peptide. The term does not refer to conventional tags like His-tags, such as His₅-tags, His₆-tags, His₇-tags, His₈-tags, His₉-tags, His₁₀-tags, His₁₁-tags, His₁₂-tags, His₁₆-tags and His₂₀-tags, Strep-tags, Avi-tags, Myc-tags, Gst-tags, JS-tags, cystein-tags, FLAG-tags or other tags known in the art, thioredoxin or maltose binding proteins (MBP). The term “tag” in contrast to the term “amino acid sequence stretch” as used herein refers to a peptide which can be useful to facilitate expression and/or affinity purification of a polypeptide, to immobilize a polypeptide to a surface or to serve as a marker or a label moiety for detection of a polypeptide e.g. by antibody binding in different ELISA assay formats as long as the function making the tag useful for one of the above listed facilitation is not caused by the positively charge of said peptide. However, the His₆-tag may, depending on the respective pH, also be positively charged, but is used as affinity purification tool as it binds to immobilized divalent cations and is not understood to be a “amino acid sequence stretch” as used herein. Preferably an amino acid sequence stretch as used herein as a length of about 6 to about 39 amino acid residues.

As used herein, the term “cationic peptide” refers preferably to a peptide having positively charged amino acid residues. Preferably a cationic peptide has a pKa-value of 9.0 or greater. Typically, at least four of the amino acid residues of the cationic peptide can be positively charged, for example, lysine or arginine. “Positively charged” refers to the side chains of the amino acid residues which have a net positive charge at about physiological conditions. The term “cationic peptide” as used herein refers also to polycationic peptides, but also includes cationic peptides which comprise for example less than 20%, preferably less than 10% positively charged amino acid residues.

The term “polycationic peptide” as used herein refers preferably to a peptide composed of mostly positively charged amino acid residues, in particular lysine and/or arginine residues. A peptide is composed of mostly positively charged amino acid residues if at least about 20, 30, 40, 50, 60, 70, 75, 80, 85, 90, 95 or about 100% of the amino acid residues are positively charged amino acid residues, in particular lysine and/or arginine residues. The amino acid residues being not positively charged amino acid residues can be neutrally charged amino acid residues and/or negatively charged amino acid residues and/or hydrophobic amino acid residues. Preferably the amino acid residues being not positively charged amino acid residues are neutrally charged amino acid residues, in particular serine and/or glycine.

The term, “antimicrobial peptide” (AMP) as used herein refers preferably to any naturally occurring peptide that has microbicidal and/or microbistatic activity on for example bacteria, viruses, fungi, yeasts, mycoplasma and protozoa. Thus, the term “antimicrobial peptide” as used herein refers in particular to any peptide having anti-bacterial, anti-fungal, anti-mycotic, anti-parasitic, anti-protozoal, anti-viral, anti-infectious, anti-infective and/or germicidal, algicidal, amoebicidal, microbicidal, bactericidal, fungicidal, parasiticidal, protozoacidal, protozoicidal properties. Preferred are anti-bacterial peptides. The antimicrobial peptide may be a member of the RNase A super family, a defensin, cathelicidin, granulysin, histatin, psoriasin, dermicidine or hepcidin. The antimicrobial peptide may be naturally occurring in insects, fish, plants, arachnids, vertebrates or mammals. Preferably the antimicrobial peptide may be naturally occurring in insects, fish, plants, arachnids, vertebrates or mammals. Preferably the antimicrobial peptide may be naturally occurring in radish, silk moth, wolf spider, frog, preferably in Xenopus laevis, Rana frogs, more preferably in Rana catesbeiana, toad, preferably Asian toad Bufo bufo gargarizans, fly, preferably in Drosophila, more preferably in Drosophila melanogaster, in Aedes aegypti, in honey bee, bumblebee, preferably in Bombus pascuorum, flesh fly, preferably in Sarcophaga peregrine, scorpion, horseshoe crab, catfish, preferably in Parasilurus asotus, cow, pig, sheep, porcine, bovine, monkey and human. As used herein, an “antimicrobial peptide” (AMP) may in particular be a peptide which is not a cationic peptide, polycationic peptide, amphiphatic peptide, sushi peptide, defensins, and hydrophobic peptide, but nevertheless exhibits antimicrobial activity.

The term “sushi peptide” as used herein refers to complement control proteins (CCP) having short consensus repeats. The sushi module of sushi peptides functions as a protein-protein interaction domain in many different proteins. Peptides containing a Sushi domain have been shown to have antimicrobial activities. Preferably, sushi peptides are naturally occurring peptides.

The term “amphiphatic peptide” as used herein refers to synthetic peptides having both hydrophilic and hydrophobic functional groups. Preferably, the term “amphiphatic peptide” as used herein refers to a peptide having a defined arrangement of hydrophilic and hydrophobic groups e.g. amphiphatic peptides may be e.g. alpha helical, having predominantly non polar side chains along one side of the helix and polar residues along the rest of its surface.

The term “hydrophobic group” as used herein refers preferably to chemical groups such as amino acid side chains which are substantially water insoluble, but soluble in an oil phase, with the solubility in the oil phase being higher than that in water or in an aqueous phase. In water, amino acid residues having a hydrophobic side chain interact with one another to generate a non-aqueous environment. Examples of amino acid residues with hydrophobic side chains are valine, isoleucine, leucine, methionine, phenylalanine, tryptophan, cysteine, alanine, tyrosine, and proline residues.

The term “hydrophobic peptide” as used herein refers to a hydrophobic peptide, which is preferably composed of mostly amino acid residues with hydrophobic groups. Such peptide is preferably composed of mostly hydrophobic amino acid residues, i.e. at least about 20, 30, 40, 50, 60, 70, 75, 80, 85, 90, 95 or at least about 100% of the amino acid residues are hydrophobic amino acid residues. The amino acid residues being not hydrophobic are preferably neutral and preferably not hydrophilic.

The term “comprising” as used herein shall not be construed as being limited to the meaning “consisting of (i.e. excluding the presence of additional other matter). Rather, “comprising” implies that optionally additional matter may be present. The term “comprising” encompasses as particularly envisioned embodiments falling within its scope “consisting of (i.e. excluding the presence of additional other matter) and “comprising but not consisting of (i.e. requiring the presence of additional other matter), with the former being more preferred.

The present invention relates to a polypeptide comprising a peptidoglycan hydrolase for use as a medicament for the treatment of chronic bacterial infections.

In particular, the present relates to a polypeptide comprising a peptidoglycan hydrolase for use as a medicament for the treatment of chronic bacterial infections, wherein the peptidoglycan hydrolase is an endolysin and wherein the chronic bacterial infection is associated with Pseudomonas (e.g. Pseudomonas aeruginosa), Escherichia (e.g. E. coli), Staphylococcus spp. (e.g. Staphylococcus aureus), Acinetobacter (e.g. Acinetobacter baumanii), Mycobacterium spp. (e.g. Mycobacterium avium, Mycobacterium tuberculos), Listeria monocytogenes and/or Salmonella bacteria.

The present invention further relates to a pharmaceutical composition comprising a polypeptide comprising a peptidoglycan hydrolase for use as a medicament for the treatment of chronic bacterial infections. In particular it relates to a pharmaceutical composition comprising a polypeptide comprising a peptidoglycan hydrolase for use as a medicament for the treatment of chronic bacterial infections, wherein the peptidoglycan hydrolase is an endolysin and wherein the chronic bacterial infection is associated with Pseudomonas (e.g. Pseudomonas aeruginosa), Escherichia (e.g. E. coli), Staphylococcus spp. (e.g. Staphylococcus aureus), Acinetobacter (e.g. Acinetobacter baumanii), Mycobacterium spp. (e.g. Mycobacterium avium, Mycobacterium tuberculosis), Listeria monocytogenes and/or Salmonella bacteria.

The polypeptide of the present invention comprising the peptidoglycan hydrolase is composed of an amino acid molecule having the amino acid sequence of an endopeptidase, N-acetyl-muramoyl-L-alanine-amidase (amidase), N-acetyl-muramidase, N-acetyl-glucosaminidase or lytic transglycosylase and thus is suitable for degrading the peptidoglycan of bacterial cell walls. The polypeptide of the present invention thus exhibits the activity of a peptidoglycan hydrolase, i.e. is capable of degrading bacterial peptidoglycan. The peptidoglycan hydrolase may be composed of an amino acid molecule having at least the amino acid sequence selected from the group of an endopeptidase, N-acetyl-muramoyl-L-alanine-amidase (amidase), N-acetyl-muramidase, N-acetyl-glucosaminidase and lytic transglycosylase, i.e. the peptidoglycan hydrolase may have more than one peptidoglycan hydrolyzing activities.

The peptidoglycan structure of a bacterial cell wall is overall largely conserved with minor modifications (Schleifer & Kandler 1972). Bacterial species have interpeptide bridges composed of different amino acids or may even lack an interpeptide bridge. In peptidoglycan structures lacking an interpeptide bridge a Diaminopimelic acid (DAP) or meso-Diaminopimelic acid (mDAP; an amino acid, representing an epsilon-carboxy derivative of lysine being a typical component of peptidoglycan) (Diaminopimelic acid is residue replaces the amino acid L-Lys and directly cross-links to the terminal amino acid D-Ala of the opposite peptide chain. Thus, there are limited types of chemical bonds available that can be hydrolyzed by peptidoglycan hydrolases. The peptidoglycan hydrolases exhibit at least one enzyme domain having an enzymatic activity as listed above. In addition the peptidoglycan hydrolases may contain at least one domain suitable for binding to the peptidoglycan and supporting the enzymatic activity of the peptidoglycan hydrolase. The binding domains are typically called cell-wall binding domains (CBD).

Preferably a domain having enzymatic activity is the N-acetylglucosaminidase that hydrolyzes the glycan component of the peptidoglycan on the reducing side of GlcNAc. Examples of peptidoglycan hydrolases exhibiting the N-acetylglucosaminidase activity are autolysins, e.g. AltA from Enterococcus faecalis (Mesnage et al., 2008) or Acm A, Acm B, Acm C, and Acm D from Lactococcus lactis (Steen et al., 2007). Further examples are endolysins, e.g. the LambdaSa2 endolysin (Pritchard et al., 2007).

Further preferably, a domain having enzymatic activity is the N-acetyl-muramidase that hydrolyzes the glycan component of the peptidoglycan on the reducing side of MurNAc., i.e. the hydrolysis of the beta-(1->4) linkages between N-acetylmuramic acid and N-acetyl-D-glucosamine residues in a peptidoglycan. Furthermore, N-acetyl-muramidase hydrolyzes N-acetyl-D-glucosamine residues in chitodextrins. The EC number of N-acetyl-muramidase is EC 3.2.1.17 with alternative names for N-acetyl-muramidase like 1,4-N-acetylmuramidase, mucopeptide, glucohydrolase, mucopeptide, N-acetylmuramoylhydrolase, muramidase, N,O-diacetylmuramidase, and peptidoglycan N-acetylmuramoylhydrolase. Examples of peptidoglycan hydrolases exhibiting the N-acetyl-muramidase activity are autolysins, exolysins, bacteriocins, and endolysins, e.g.: Cpl −1 endolysin (Garcia et al., 1987), B30 endolysin (Pritchard et al., 2004), Cp1-7 (Garcia et al., 1990).

Further preferably, a domain having enzymatic activity is the lytic transglycosylase that hydrolyzes the same bond as muraminidases, but form N-acetyl-1,6-anhydro-muramyl intermediates during hydrolysis, i.e. the hydrolysis of the b(1 ->4) linkages between N-acetylmuramyl and N-acetylglucosaminyl residues of the peptidoglyccan. The reaction includes forming a N-acetyl-1,6-anhydro-muramyl moiety residue during glycosidic cleavage. The EC number of lytic transglycosylase is EC 4.2.2.n1—Peptidoglycan lytic exotransglycosylase with alternative names for lytic transglycosylase like peptidoglycan lytic exotransglycosylase and exomuramidase. Examples of peptidoglycan hydrolases exhibiting the lytic transglycosylase activity are phage lambda endolysin (Taylor and Gorazdowska, 1974) and phage Phi K Z gp144 endolysin (Paradis-Bleau et al., 2007).

Further preferably, a domain having enzymatic activity is the N-acetylmuramoyl-L-alanine amidase that hydrolyzes the amide bond between the glycan moiety (MurNAc) and the peptide moiety (L-alanine) of the cell wall. The EC number of N-acetylmuramoyl-L-alanine amidase is EC 3.5.1.28 with alternative names for N-acetylmuramoyl-L-alanine amidase like amidase, acetylmuramyl-L-alanine amidase; N-acetylmuramyl-L-alanine amidase; N-acylmuramyl-L-alanine amidase; acetylmuramoyl-alanine amidase; N-acetylmuramic acid L-alanine amidase; acetylmuramyl-alanine amidase; N-acetylmuramylalanine amidase; murein hydrolase; N-acetylmuramoyl-L-alanine amidase type I; N-acetylmuramoyl-L-alanine amidase type II. Examples of peptidoglycan hydrolases exhibiting the N-acetylmuramoyl-L-alanine amidase activity are amidase domain of the staphylococcal phage F11 endolysin (Navarre et al., 1999), LysK (Becker et al., 2009a; Donovan et al., 2009), Listeria phage endolysin Ply511 (Loessner et al., 1995b) and PlyPSA (Korndorfer et al., 2006).

Further preferably, a domain having enzymatic activity is the endopeptidase that hydrolyzes peptide bonds between two amino acids. Cleavage may occur in the stem peptide, e.g. Ply500 and Ply118 L-alanyl-D-glutamate endolysins (Loessner et al., 1995b) or in the interpeptide bridge, e.g., F 11 D-alanyl-glycyl endolysin (Navarre et al., 1999) or the lysostaphin bacteriocin/exolysin. The EC number of endopeptidase is EC 3.4.25.74 in case of Lysostaphin, and EC 3.4.24 in case of Plyll8. An alternative name for endopeptidase is metallopeptidase. Examples of peptidoglycan hydrolases exhibiting the endopeptidase activity are. Ply500, Ply118, L-alanyl-D-glutamate endolysins (Loessner et al., 1995b), Ply PSA, LysK, Lambda SaII, F11 D-alanyl-glycyl endolysin (Navarre et al., 1999), B30, lysostaphin.

The peptidoglycan degrading activity of a polypeptide having peptidoglycan hydrolase activity on Gram-negative and Gram-positive bacteria can be measured by assays well known in the art, e.g. by muralytic assays in which the outer membrane of Gram-negative bacteria is permeabilized or removed (e.g. with chloroform) to allow the putative enzyme access to the peptidoglycan layer. If the enzyme is active, degradation of the peptidoglycan layer will lead to a drop of turbidity, which can be measured photometrically (see for example Briers et al., J. Biochem. Biophys Methods 70: 531-533, (2007) or Schmelcher et al., Bacteriophage endolysins as novel antimicrobials. Schmelcher M, Donovan D M, Loessner M J. Future Microbiol. 2012 October; 7(10):1147-7).

In a preferred embodiment, the polypeptide of to the present invention has peptidoglycan hydrolase activity on Gram-negative bacteria of bacterial groups, families, genera or species comprising strains pathogenic for humans or animals like Enterobacteriaceae, in particular Escherichia, Salmonella, Shigella, Citrobacter, Edwardsiella, Enterobacter, Hafnia, Klebsiella, Morganella, Proteus, Providencia, Serratia, and Yersinia; Pseudomonadaceae, in particular Burkholderia, Pseudomonas, Stenotrophomonas, Shewanella, Sphingomonas and Comamonas; Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Spirochaetaceae, in particular Treponema and Borrelia; Leptospiraceae, Campylobacter, Helicobacter, Spirillum, Streptobacillus, Bacteroidaceae, in particular Bacteroides, Fusobacterium, Prevotella and Porphyromonas; and Acinetobacter, in particular A. baumanii.

In a preferred embodiment, the polypeptide of to the present invention has peptidoglycan hydrolase activity on Gram-positive bacteria of bacterial groups, families, genera or species comprising strains pathogenic for humans or animals like Listeria monocytogenes, Staphylococcus aureus, Enterococcus faecalis, Enterococcus faecium, Streptococcus pneumoniae, Streptococcus pyogenes, Streptococcus uberis, Streptococcus agalacticae, Streptococcus mutans, Streptococcus equi, Clostridium difficile, Clostridium botulinum, Clostridium tetani, Clostridium perfringens, Bacillus anthracis, Bacillus cereus, Propionibacterium acnes, Mycobacterium avium, Mycobacterium abscessus, Mycobacterium tuberculosis, Corynebacterium diphteriae, Mycoplasma pneumoniae, Actinomyces.

Preferably, the peptidoglycan hydrolase is an endolysin, an autolysin or a bacteriocin.

Thus, in another aspect the present invention relates to a polypeptide comprising an endolysin, an autolysin or a bacteriocin for use as a medicament for the treatment of chronic bacterial infections.

Thus, in another aspect the present invention further relates to a pharmaceutical composition comprising a polypeptide comprising an endolysin, an autolysin or a bacteriocin in a method of treatment of chronic bacterial infections.

Preferred endolysins are PhiV10p30 of phage (I)V10 (Sequence analysis of Escherichia coli O157:H7 bacteriophage PhiV10 and identification of a phage-encoded immunity protein that modifies the O157 antigen. Perry L L, SanMiguel P, Minocha U, Terekhov A I, Shroyer M L, Farris L A, Bright N, Reuhs B L, Applegate B M. FEMS Microbiol Lett. 2009 March; 292(2):182-6); STM0907.Fels0 of phage FELS-1 (Nature. 2001 Oct. 25; 413(6858):852-6. Complete genome sequence of Salmonella enterica serovar Typhimurium LT2. McClelland M1, Sanderson K E, Spieth J, Clifton S W, Latreille P, Courtney L, Porwollik S, Ali J, Dante M, Du F, Hou S, Layman D, Leonard S, Nguyen C, Scott K, Holmes A, Grewal N, Mulvaney E, Ryan E, Sun H, Florea L, Miller W, Stoneking T, Nhan M, Waterston R, Wilson R K); epsilon15p25 of phage ε15 (Virology. 2007 Dec. 20; 369(2):234-44. Epub 2007 Sep. 7. The genome of epsilon15, a serotype-converting, Group E1 Salmonella enterica-specific bacteriophage. Kropinski AM1, Kovalyova I V, Billington S J, Patrick A N, Butts B D, Guichard J A, Pitcher T J, Guthrie C C, Sydlaske A D, Barnhill L M, Havens K A, Day K R, Falk D R, McConnell M R); YuA20 of phage YUA (NCBI Reference Sequence: YP_001595885.1 and J Bacteriol. 2008 February; 190(4):1429-35. The genome and structural proteome of YuA, a new Pseudomonas aeruginosa phage resembling M6. Ceyssens PJ1, Mesyanzhinov V, Sykilinda N, Briers Y, Roucourt B, Lavigne R, Robben J, Domashin A, Miroshnikov K, Volckaert G, Hertveldt K); ORF23 of phage B3 (Complete genomic sequence of bacteriophage B3, a Mu-like phage of Pseudomonas aeruginosa. Braid M D, Silhavy J L, Kitts C L, Cano R J, Howe M M. J Bacteriol. 2004 October; 186(19):6560-74); BcepMu22 of phage BcepMu (J Mol Biol. 2004 Jun. 25; 340(1):49-65. Burkholderia cenocepacia phage BcepMu and a family of Mu-like phages encoding potential pathogenesis factors. Summer EJ1, Gonzalez C F, Carlisle T, Mebane L M, Cass A M, Savva C G, LiPuma J, Young R); F116p62 of phage F116 (Gene. 2005 Feb. 14; 346:187-94. The genome of the Pseudomonas aeruginosa generalized transducing bacteriophage F116. Byrne M1, Kropinski A M.); STM2715.S.Fels2 of phage Fels2 (Nature. 2001 Oct. 25; 413(6858):852-6. Complete genome sequence of Salmonella enterica serovar Typhimurium LT2. McClelland M1, Sanderson K E, Spieth J, Clifton S W, Latreille P, Courtney L, Porwollik S, Ali J, Dante M, Du F, Hou S, Layman D, Leonard S, Nguyen C, Scott K, Holmes A, Grewal N, Mulvaney E, Ryan E, Sun H, Florea L, Miller W, Stoneking T, Nhan M, Waterston R, Wilson R K); gp76 of phage ES18 (C asj ens, S. R., Gilcre ase, E. B., Winn-Stapley, D. A., Schicklmaier, P., Schmieger, H., Pedulla, M. L., Ford, M. E., Houtz, J. M., Hatfull, G. F. and Hendrix, R. W. The generalized transducing Salmonella bacteriophage ES18: complete genome sequence and DNA packaging strategy J. Bacteriol. 187 (3), 1091-1104 (2005)); SPSV3_gp23 of phage SETP3 (J Med Microbiol. 2009 January; 58(Pt 1):86-93. Characterization of bacteriophages used in the Salmonella enterica serovar Enteritidis phage-typing scheme. De Lappe N1, Doran G, O'Connor J, O'Hare C, Cormican M); phi32_17 of phage ΦECO32 (Genomic and proteomic analysis of phiEco32, a novel Escherichia coli bacteriophage. Savalia D, Westblade L F, Goel M, Florens L, Kemp P, Akulenko N, Pavlova O, Padovan J C, Chait B T, Washburn M P, Ackermann H W, Mushegian A, Gabisonia T, Molineux I, Severinov K. J Mol Biol. 2008 Mar. 28; 377(3):774-89); HK022p54 of phage HK022 (J Mol Biol. 2000 May 26; 299(1):27-51. Genomic sequences of bacteriophages HK97 and HK022: pervasive genetic mosaicism in the lambdoid bacteriophages. Juhala RJ1, Ford M E, Duda R L, Youlton A, Hatfull G F, Hendrix R W); HK97p58 of phage HK97 (J Mol Biol. 2000 May 26; 299(1):27-51. Genomic sequences of bacteriophages HK97 and HK022: pervasive genetic mosaicism in the lambdoid bacteriophages. Juhala R J1, Ford M E, Duda R L, Youlton A, Hatfull G F, Hendrix R W); HK620p36 of phage HK620 (Nucleotide sequence of coliphage HK620 and the evolution of lambdoid phages. Clark A J, Inwood W, Cloutier T, Dhillon T S. J Mol Biol. 2001 Aug. 24; 311(4):657-79); VIP0007 of phage E1 (Molecular characterization of the Salmonella enterica serovar Typhi Vi-typing bacteriophage E1. Pickard D, Thomson N R, Baker S, Wain J, Pardo M, Goulding D, Hamlin N, Choudhary J, Threfall J, Dougan G. J Bacteriol. 2008 April; 190(7):2580-7); Sf6p62 of phage SF6 (J Mol Biol. 2004 May 28; 339(2):379-94. The chromosome of Shigella flexneri bacteriophage Sf6: complete nucleotide sequence, genetic mosaicism, and DNA packaging. Casjens S1, Winn-Stapley D A, Gilcrease E B, Morona R, Kühlewein C, Chua J E, Manning P A, Inwood W, Clark A J); R (SfVp40) of phage SFV (J Bacteriol. 2002 April; 184(7):1974-87. Complete genomic sequence of SfV, a serotype-converting temperate bacteriophage of Shigella flexneri. Allison GE1, Angeles D, Tran-Dinh N, Verma N K); gp22 of phage BCEPC6B (Summer, E. J., Christian, B. N., Collins, J., Morrison, W., Patel, P., Wells, W., Mebane, L., Gonzalez, C. F. and Young, R. F. GenBank: AAT38381.1); Nazgul38 of phage BCEPNAZGUL (Summer, E. J., Peek, M. L., Haliburton, J. R., Hall, E., Heusinkveld, K., Simser, J., No, E. G., Gonzalez, C. F. and Young, R. F. NCBI Reference Sequence: NP_918971.2); K (P2p09) of phage P2 (Christie,G. E., Haggard-Ljungquist,E. and Calendar,R. NCBI Reference Sequence: NP_046765.1); K (Wphi09) of phage Wφ (Esposito, D., Schmidt, B. J., Bloom, F. R. and Christie, G. E. GenBank: AAN28227.1); rv5_gp085 of phage RV5 (Virol J. 2013 Mar 6; 10:76. The host-range, genomics and proteomics of Escherichia coli O157:H7 bacteriophage rV5. Kropinski AM1, Waddell T, Meng J, Franklin K, Ackermann H W, Ahmed R, Mazzocco A, Yates J 3rd, Lingohr E J, Johnson R P); EpJS98_gp116 of phage J598 (Zuber, S., Ngom-Bru, C., Barretto, C., Bruttin, A., Brussow, H. and Denou, E. Genome analysis of phage J598 defines a fourth major subgroup of T4-like phages in Escherichia coli J. Bacteriol. 189 (22), 8206-8214 (2007)); gp3.5 of phage 13A (Savalia, D., Severinov, K. and Molineux, I. NCBI Reference Sequence: YP_002003950.1); gp3.5 of phage BA14 (Savalia, D., Severinov, K. and Molineux, I. GenBank: ACF15743.1); gp3.5 of phage ECODS1 (Savalia, D., Severinov, K. and Molineux, I. GenBank: ACF15800.1); CKV1F gp16 of phage K1F (Scholl, D. and Merril, C. The Genome of Bacteriophage K1F, a T7-Like Phage That Has Acquired the Ability To Replicate on K1 Strains of Escherichia coli J. Bacteriol. 187 (24), 8499-8503 (2005)); T3p18 of phage T3 (Pajunen, M. I., Elizondo, M. R., Skurnik, M., Kieleczawa, J. and Molineux, I. J. Complete nucleotide sequence and likely recombinatorial origin of bacteriophage T3 J. Mol. Biol. 319 (5), 1115-1132); gh-1p12 of phage GH-1 (Kovalyova, I .V. and Kropinski, A. M. The complete genomic sequence of lytic bacteriophage gh-1 infecting Pseudomonas putida—evidence for close relationship to the T7 group Virology 311 (2), 305-315 (2003)); gp3.5 of phage K11 (Savalia, D., Severinov, K. and Molineux, I. NCBI Reference Sequence: YP_002003804.1); ORF12 of phage ΦCTX (Nakayama, K., Kanaya, S., Ohnishi, M., Terawaki, Y. and Hayashi, T. The complete nucleotide sequence of phi CTX, a cytotoxin-converting phage of Pseudomonas aeruginosa: implications for phage evolution and horizontal gene transfer via bacteriophages Mol. Microbiol. 31 (2), 399-419 (1999)); Bcep43-27 of phage BCEP43 (Summer, E. J., Gonzalez, C. F., Bomer, M., Carlile, T., Embry, A., Kucherka, A. M., Lee, J., Mebane, L., Morrison, W. C., Mark, L., King, M. D., LiPuma, J. J., Vidaver, A. K. and Young, R. Divergence and mosaicism among virulent soil phages of the Burkholderia cepacia complex J. Bacteriol. 188 (1), 255-268 (2006)); Bcep781-27 of phage BCEP781 (Summer, E. J., Gonzalez, C. F., Bomer, M., Carlile, T., Embry, A., Kucherka, A. M., Lee, J., Mebane, L., Morrison,W. C., Mark, L., King, M. D., LiPuma, J. J., Vidaver, A. K. and Young, R. Divergence and mosaicism among virulent soil phages of the Burkholderia cepacia complex J. Bacteriol. 188 (1), 255-268 (2006)); Bcep1-28 of phage BCEP1 (Summer, E. J., Gonzalez, C.F., Bomer, M., Carlile, T., Embry, A., Kucherka, A. M., Lee, J., Mebane, L., Morrison, W. C., Mark, L., King, M. D., LiPuma, J. J., Vidaver, A. K. and Young, R. Divergence and mosaicism among virulent soil phages of the Burkholderia cepacia complex J. Bacteriol. 188 (1), 255-268 (2006)); BcepNY3gene26 of phage BCEPNY3 (GenBank: ABR10561.1 Summer, E. J., Orchard, R. C., Attenhofer, K., Coffey, A., Gill, J. J., Gonzalez, C. F. and Young, R.); gp45 of phage ΦE12-2 (NCBI Reference Sequence: YP_001111195.1 DeShazer, D., Ronning, C. M., Brinkac, L. M. and Nierman, W. C.); gp28 of phage Φ52237 (DeShazer, D and Nierman, W.C. NCBI Reference Sequence: YP_293741.1 DeShazer, D., Ronning, C. M., Brinkac, L. M. and Nierman, W. C.); P27p30 of phage ΦP27 (Recktenwald, J. and Schmidt, H. The nucleotide sequence of Shiga toxin (Stx) 2e-encoding phage phiP27 is not related to other Stx phage genomes, but the modular genetic structure is conserved Infect. Immun. 70 (4), 1896-1908 (2002)); RB49p102 of phage RB49 (Monod, C., Repoila, F., Kutateladze, M., Tetart, F. and Krisch, H. M. The genome of the pseudo T-even bacteriophages, a diverse group that resembles T4 J. Mol. Biol. 267 (2), 237-249 (1997)); phil-p102 of phage Φ1)1 (Arbiol, C., Comeau, A. M., Kutateladze, M., Adamia, R. and Krisch, H. M. Mobile regulatory cassettes mediate modular shuffling in t4-type phage genomes Genome Biol Evol 2010, 140-152 (2010)); lys (T5.040) of phage T5 (NCBI Reference Sequence: YP_006868.1 Ksenzenko, V. N., Kaliman, A. V., Krutilina, A. I. and Shlyapnikov, M. G.); YP_001956952.1 of phage 201phi2-1 (Thomas, J. A., Rolando, M. R., Carroll, C. A., Shen, P. S., Belnap, D. M., Weintraub, S. T., Serwer, P. and Hardies, S. C. Characterization of Pseudomonas chlororaphis myovirus 20lvarphi2-1 via genomic sequencing, mass spectrometry, and electron microscopy Virology 376 (2), 330-338 (2008)); Aehlp339 of phage Aehl (NCBI Reference Sequence: NP_944217.1 Petrov, V., Nolan, J., Bertrand, C., Letarov, A. V., Krisch, H. M. and Karam, J. D); YYZgp45 of phage YYZ-2008 (GenBank: ACI32381.1 Zhang, Y., Laing, C. R., Kropinski, A. and Gannon, V. J. P.).

Also preferred is the endolysin of the Pseudomonas aeruginosa phages ΦKZ, gp144 (J Biol Chem. 2008 Mar. 14; 283(11):7242-50. Structure of the bacteriophage phi KZ lytic transglycosylase gp144. Fokine A, Miroshnikov K A, Shneider M M, Mesyanzhinov V V, Rossmann M G.), and EL, EL188 (Mol Microbiol. 2007 September; 65(5):1334-44. Muralytic activity and modular structure of the endolysins of Pseudomonas aeruginosa bacteriophages phiKZ and E L. Briers Y1, Volckaert G, Cornelissen A, Lagaert S, Michiels C W, Hertveldt K, Lavigne R), of the phage LUZ24 (NCBI Reference Sequence: YP_001671940.1) as well as of the E. coli phage N4gp61 (J Mol Biol. 2007 Feb. 16; 366(2):406-19. Coliphage N4 N-acetylmuramidase defines a new family of murein hydrolases. StojkoviéEA1, Rothman-Denes L B), STM0016 endolysin (NCBI Reference Sequence: NP_459021.1), PSP3 endolysin (NP_958065.1) and endolysin of Salmonella enteritis phage PVPSE1 (PVP-SE1gp146 (YP_004893953.1)).

Further preferred endolysins are Listeria phage endolysins PlyA118 (NCBI Reference Sequence: YP_008666952.1), PlyA500 (NCBI Reference Sequence: YP_001468411.1), PlyPSA (GenBank: CAC85577.1 and J Mol Biol. 2006 Dec. 8; 364(4):678-89. The crystal structure of the bacteriophage PSA endolysin reveals a unique fold responsible for specific recognition of Listeria cell walls. Korndörfer IP1, Danzer J, Schmelcher M, Zimmer M, Skerra A, Loessner M J), PlyA511 (NCBI Reference Sequence: YP_001468459.1), PlyP35 (GenBank: AAY53213.1), PlyP40 (NCBI Reference Sequence: YP_002261442.1), Staphylococcal phage Phi 11 endolysin (Lytic activity of recombinant bacteriophage phi11 and phi12 endolysins on whole cells and biofilms of Staphylococcus aureus. Sass P, Bierbaum G. Appl Environ Microbiol. 2007 January; 73(1):347-52), Phi MR11 endolysin (NCBI Reference Sequence: YP_001604156.1), LysK (The recombinant phage lysin LysK has a broad spectrum of lytic activity against clinically relevant staphylococci, including methicillin-resistant Staphylococcus aureus. O'Flaherty S, Coffey A, Meaney W, Fitzgerald G F, Ross R P. J Bacteriol. 2005 October; 187(20):7161-4), Clostridium perfringens PlyS9 (WO2010003943 (A1; Bacteriophage. 2012 Apr. 1; 2(2):89-97. Inducible Clostridium perfringens bacteriophages ΦS9 and ΦS63: Different genome structures and a fully functional sigK intervening element. Kim KP1, Born Y, Lurz R, Eichenseher F, Zimmer M, Loessner M J, Klumpp J.), Ply3626 (Zimmer, M., Scherer, S. and Loessner, M. J. Genomic analysis of Clostridium perfringens bacteriophage phi3626, which integrates into guaA and possibly affects sporulation J. Bacteriol. 184 (16), 4359-4368 (2002)), Clostridium difficile: CD27L endolysin (J Bacteriol. 2008 October; 190(20):6734-40. Molecular characterization of a Clostridium difficile bacteriophage and its cloned biologically active endolysin. Mayer MJ1, Narbad A, Gasson M J), Streptococcus: B30 endolysin (The bifunctional peptidoglycan lysin of Streptococcus agalactiae bacteriophage B30. Pritchard D G, Dong S, Baker J R, Engler J A. Microbiology. 2004 July; 150(Pt 7):2079-87), phage Dp-1 encoded Pal amidase (J Biol Chem. 2004 Oct. 15; 279(42):43697-707. Structural and thermodynamic characterization of Pal, a phage natural chimeric lysin active against pneumococci. Varea J l, Monterroso B, Sáiz J L, López-Zumel C, García J L, Laynez J, Garcia P, Menéndez M.), C1 endolysin PlyC (PlyC: a multimeric bacteriophage lysin. Nelson D, Schuch R, Chahales P, Zhu S, Fischetti V A. Proc Natl Acad Sci U S A. 2006 Jul. 11; 103(28):10765-70), Cp1-1 endolysin (Gene. 1990 Jan. 31; 86(1):81-8. Modular organization of the lytic enzymes of Streptococcus pneumoniae and its bacteriophages. García P l, García J L, García E, Sánchez-Puelles J M, López R.), PlyGBS (Antimicrob Agents Chemother. 2005 January; 49 (l):111-7. Removal of group B streptococci colonizing the vagina and oropharynx of mice with a bacteriophage lytic enzyme. Cheng Q1, Nelson D, Zhu S, Fischetti V A.), Enterococccus: PlyV12 (J Bacteriol. 2004 July; 186(14):4808-12. Identification of a broadly active phage lytic enzyme with lethal activity against antibiotic-resistant Enterococcus faecalis and Enterococcus faecium. Yoong P1, Schuch R, Nelson D, Fischetti V A.), Bacillus anthracis: Phage gamma endolysin PlyG (Nature. 2002 Aug. 22; 418(6900):884-9. A bacteriolytic agent that detects and kills Bacillus anthracis. Schuch R, Nelson D, Fischetti V A.).

More preferred endolysins are listed as SEQ ID NO: 1-11.

Preferred autolysins are described in: Bacterial peptidoglycan (murein) hydrolases. Vollmer W, Joris B, Charlier P, Foster S. FEMS Microbiol Rev. 2008 March; 32(2):259-86. Epub 2008 Feb. 11. Review. An example of a preferred autolysin is the AtlA Autolysine.

Preferred bacteriocins are Lysostaphin (degrading Staphylococcus cell walls), Mutanolysin (degrading Streptococcus cell walls) and Enterolysin (degrading Enterococcus cell walls). A more preferred lysostaphin is listed as SEQ ID NO: 12.

A polypeptide of to the present invention may comprise additionally to the peptide hydrolase at least one amino acid sequence stretch, e.g. an amino acid sequence stretch selected from the group consisting of amphiphatic peptide, cationic peptide, polycationic peptide, hydrophobic peptide, or naturally occurring antimicrobial peptide, like sushi peptide and defensin. This additional at least one amino acid sequence stretch may be present at any position in the polypeptide of to the present invention, as long as it will not disrupt the peptidoglycan hydrolyzing activity, but is preferably present at the termini, i.e. in the N- and/or C-terminal region of the polypeptide of to the present invention. Such additional amino acid sequence stretch may be fused directly, or via a peptide linker, to the rest of the polypeptide, e.g. the peptidoglycan hydrolase.

More preferred are cationic and/or polycationic amino acid sequence stretches comprising at least one motive according to SEQ ID NO: 13 (KRKKRK). In particular cationic amino acid sequence stretches comprising at least 2, 3, 4, 5, 6, 7, 8, 9, 10, 11, 12, 13, 14, 15, 16 or 17 motives according to SEQ ID NO: 13 (KRKKRK) are preferred. More preferred are cationic peptide stretches comprising at least one KRK motive (lys-arg-lys), preferable at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 KRK motives.

In another preferred embodiment of the present invention the cationic amino acid sequence stretch comprises beside the positively charged amino acid residues, in particular lysine and/or arginine residues, neutrally charged amino acid residues, in particular glycine and/or serine residues. Preferred are cationic amino acid sequence stretches consisting of about 70% to about 100%, or about 80% to about 95%, or about 85% to about 90% positively charged amino acid residues, in particular lysine, arginine and/or histidine residues, more preferably lysine and/or arginine residues and of about 0% to about 30%, or about 5% to about 20%, or about 10% to about 20% neutrally charged amino acid residues, in particular glycine and/or serine residues. Preferred are amino acid sequence stretches consisting of about 4% to about 8% serine residues, of about 33% to about 36% arginine residues and of about 56% to about 63% lysine residues. Especially preferred are amino acid sequence stretches comprising at least one motive according to SEQ ID NO: 14 (KRXKR), wherein X is any other amino acid than lysine, arginine and histidine. Especially preferred are polypeptide stretches comprising at least one motive according to SEQ ID NO: 15 (KRSKR). More preferred are cationic stretches comprising at least about 2, 3, 4, 5, 6, 7, 8, 9, 10, 11, 12, 13, 14, 15, 16, 17, 18, 19 or at least about 20 motives according to SEQ ID NO: 14 (KRXKR) or SEQ ID NO: 15 (KRSKR).

Also preferred are amino acid sequence stretches consisting of about 9 to about 16% glycine residues, of about 4 to about 11% serine residues, of about 26 to about 32% arginine residues and of about 47 to about 55% lysine residues. Especially preferred are amino acid sequence stretches comprising at least one motive according to SEQ ID NO: 16 (KRGSG). More preferred are cationic stretches comprising at least about 2, 3, 4, 5, 6, 7, 8, 9, 10, 11, 12, 13, 14, 15, 16, 17, 18, 19 or at least bout 20 motives according to SEQ ID NO: 16 (KRGSG).

In another preferred embodiment of the present invention such cationic amino acid sequence stretch comprises beside the positively charged amino acid residues, in particular lysine and/or arginine residues, hydrophobic amino acid residues, in particular valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues. Preferred are cationic amino acid sequence stretches consisting of about 70% to about 100%, or about 80% to about 95%, or about 85% to about 90% positively charged amino acid residues, in particular lysine and/or arginine residues and of about 0% to about 30%, or about 5% to about 20%, or about 10% to about 20% hydrophobic amino acid residues, valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues.

Examples for cationic and polycationic amino acid sequence stretches are listed in the following table:

TABLE 2

| amino acid sequence stretch | length | SEQ ID NO: |
|---|---|---|
| KRKKRK | 6 | SEQ ID NO: 13 |
| KRKKRKKRK | 9 | SEQ ID NO: 17 |
| RRRRRRRRR | 9 | SEQ ID NO: 18 |
| KKKKKKKK | 8 | SEQ ID NO: 19 |
| KRKKRKKRKK | 10 | SEQ ID NO: 20 |
| KRKKRKKRKKRK | 12 | SEQ ID NO: 21 |
| KRKKRKKRKKRKKR | 14 | SEQ ID NO: 22 |
| KKKKKKKKKKKKKKKK | 16 | SEQ ID NO: 23 |
| KRKKRKKRKKRKKRKKRK | 18 | SEQ ID NO: 24 |
| KRKKRKKRKKRKKRKKRKK | 19 | SEQ ID NO: 25 |
| RRRRRRRRRRRRRRRRRRR | 19 | SEQ ID NO: 26 |
| KKKKKKKKKKKKKKKKKKK | 19 | SEQ ID NO: 27 |
| KRKKRKKRKRSKRKKRKKRK | 20 | SEQ ID NO: 28 |
| KRKKRKKRKRSKRKKRKKRKK | 21 | SEQ ID NO: 29 |
| KRKKRKKRKKRKKRKKRKKRK | 21 | SEQ ID NO: 30 |
| KRKKRKKRKRGSGKRKKRKKRK | 22 | SEQ ID NO: 31 |
| KRKKRKKRKRGSGSGKRKKRKKRK | 24 | SEQ ID NO: 32 |
| KRKKRKKRKKRKKRKKRKKRKKRKK | 25 | SEQ ID NO: 33 |
| KRKKRKKRKRSKRKKRKKRKRSKRKKRKKRK | 31 | SEQ ID NO: 34 |
| KRKKRKKRKRGSGSGKRKKRKKRKGSGSGKRKKRKKRK | 38 | SEQ ID NO: 35 |
| KRKKRKKRKKRKKRKKRKKRKKRKKRKKRKKRKKRKKRK | 39 | SEQ ID NO: 36 |
| KRKKRKKRKRSKRKKRKKRKRSKRKKRKKRKRSKRKKRKKRK | 42 | SEQ ID NO: 37 |

In a further aspect of the present invention at least one of the additional amino acid sequence stretches is an antimicrobial peptide, which comprises a positive net charge and around 50% hydrophobic amino acids. The antimicrobial peptides are amphiphatic with a length of about 12 to about 50 amino acid residues. The antimicrobial peptides are naturally occurring in insects, fish, plants, arachnids, vertebrates or mammals. Preferably the antimicrobial peptide may be naturally occurring in radish, silk moth, wolf spider, frog, preferably in Xenopus laevis, Rana frogs, more preferably in Rana catesbeiana, toad, preferably Asian toad Bufo bufo gargarizans, fly, preferably in Drosophila, more preferably in Drosophila melanogaster, in Aedes aegypti, in honey bee, bumblebee, preferably in Bombus pascuorum, flesh fly, preferably in Sarcophaga peregrine, scorpion, horseshoe crab, catfish, preferably in Parasilurus asotus, cow, pig, sheep, porcine, bovine, monkey and human.

In another preferred embodiment of the present invention the antimicrobial amino acid sequence stretches consist of about 0% to about 5%, or about 0% to about 35%, or about 10% to about 35% or about 15% to about 45%, or about 20% to about 45% positively charged amino acid residues, in particular lysine and/or arginine residues and of about 50% to about 80%, or about 60% to about 80%, or about 55% to about 75%, or about 70% to about 90% hydrophobic amino acid residues, valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues.

In another preferred embodiment of the present invention the antimicrobial amino acid sequence stretches consist of about 4% to about 58% positively charged amino acid residues, in particular lysine and/or arginine residues and of about 33% to about 89% hydrophobic amino acid residues, valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues.

Examples for antimicrobial amino acid sequences which may be used in carrying out the present invention are listed in the following table.

TABLE 3

| Peptide | Sequence | SEQ ID NO |
|---|---|---|
| LL-37 | LLGDFFRKSKEKIGKEFKRIVQRIKDFLRNLVPR | SEQ ID NO: 38 |
| | TES | |
| SMAP-29 | RGLRRLGRKIAHGVKKYGPTVLRIIRIAG | SEQ ID NO: 39 |
| Indolicidin | ILPWKWPWWPWRR | SEQ ID NO: 40 |
| Protegrin | RGGRLCYCRRRFCVCVGR | SEQ ID NO: 41 |
| Cecropin P1 | SWLSKTAKKLENSAKKRISEGIAIAIQGGPR | SEQ ID NO: 42 |
| Magainin | GIGKFLHSAKKFGKAFVGEIMNS | SEQ ID NO: 43 |
| Pleurocidin | GWGSFFKKAAHVGKHVGKAALTHYL | SEQ ID NO: 44 |
| Cecropin A | GGLKKLGKKLEGAGKRVFNAAEKALPVVAGAKAL | SEQ ID NO: 45 |
| (A. aegypti) | RK | |
| Cecropin A | GWLKKIGKKIERVGQHTRDATIQGLGIPQQAANV | |
| (D. | AATARG | SEQ ID NO: 46 |
| melanogaster) | | |
| Buforin II | TRSSRAGLQFPVGRVHRLLRK | SEQ ID NO: 47 |
| Sarcotoxin IA | GWLKKIGKKIERVGQHTRDATIQGLGIAQQAANV | SEQ ID NO: 48 |
| | AATAR | |
| Apidaecin | ANRPVYIPPPRPPHPRL | SEQ ID NO: 49 |
| Ascaphine 5 | GIKDWIKGAAKKLIKTVASHIANQ | SEQ ID NO: 50 |
| Nigrocine 2 | GLLSKVLGVGKKVLCGVSGLVC | SEQ ID NO: 51 |
| Pseudin 1 | GLNTLKKVFQGLHEAIKLINNHVQ | SEQ ID NO: 52 |
| Ranalexin | FLGGLIVPAMICAVTKKC | SEQ ID NO: 53 |
| Melittin | GIGAVLKVLTTGLPALISWIKRKRQQ | SEQ ID NO: 54 |
| Lycotoxin 1 | IWLTALKFLGKHAAKKLAKQQLSKL | SEQ ID NO: 55 |
| Parasin 1 | KGRGKQGGKVRAKAKTRSS | SEQ ID NO: 56 |
| Buforin I | AGRGKQGGKVRAKAKTRSSRAGLQFPVGRVHRLL | SEQ ID NO: 57 |
| | RKGNY | |
| Dermaseptin 1 | ALWKTMLKKLGTMALHAGKAALGAAADTISQGTQ | SEQ ID NO: 58 |
| Bactenecin 1 | RLCRIVVIRVCR | SEQ ID NO: 59 |
| Thanatin | GSKKPVPIIYCNRRTGKCQRM | SEQ ID NO: 60 |
| Brevinin 11 | VNPIILGVLPKVCLITKKC | SEQ ID NO: 61 |
| Ranateurin 1 | SMLSVLKNLGKVGLGFVACKINIKQC | SEQ ID NO: 62 |
| Esculentin 1 | GIFSKLGRKKIKNLLISGLKNVGKEVGMDVVRTG | SEQ ID NO: 63 |
| | IKIAGCKIKGEC | |
| Tachyplesin | RWCFRVCYRGICYRKCR | SEQ ID NO: 64 |
| Androctonin | RSVCRQIKICRRRGGCYYKCTNRPY | SEQ ID NO: 65 |
| alpha-defensin | DCYCRIPACIAGERRYGTCIYQGRLWAFCC | SEQ ID NO: 66 |
| beta-defensin | NPVSCVRNKGICVPIRCPGSMKQIGTCVGRAVKC | SEQ ID NO: 67 |
| | CRKK | |
| theta-defensin | GFCRCLCRRGVCRCICTR | SEQ ID NO: 68 |
| defensin | ATCDLLSGTGINHSACAAHCLLRGNRGGYCNGKA | SEQ ID NO: 69 |
| (sapecin A) | VCVCRN | |
| Thionin | TICCPSIVARSNFNVCRIPGIPEAICATYTGCII | SEQ ID NO: 70 |
| (crambin) | IPGATCPGDYAN | |
| defensin from | QKLCQRPSGTWSGVCGNNNACKNQCIRLEKARHG | SEQ ID NO: 71 |
| radish | SCNYVFPAHCICYFPC | |
| Drosomycin | DCLSGRYKGPCAVWDNETCRRVCKEEGRSSGHCS | SEQ ID NO: 72 |
| | PSLKCWCEGC | |
| Hepcidin | DTHFPICIFCCGCCHRSKCGMCCKT | SEQ ID NO: 73 |
| Bac 5 | RFRPPIRRPPIRPPFYPPFRPPIRPPIFPPIRPP | SEQ ID NO: 74 |
| | FRPPLGRPFP | |
| PR-39 | RRRPRPPYLPRPRPPPFFPPRLPPRIPPGFPPRF | SEQ ID NO: 75 |
| | PPRFP | |

The amino acid sequence stretch may be selected for example from LL-37, SMAP-29, Indolicidin, Protegrin, Cecropin P1, Magainin, Pleurocidin, Cecropin A (A.aegypti), Cecropin A (D. melanogaster), Buforin II, Sarcotoxin IA, Apidaecin, Ascaphine 5, Nigrocine 2, Pseudin 1, Ranalexin, Melittin, Lycotoxin 1, Parasin 1, Buforin I, Dermaseptin 1, Bactenecin 1, Thanatin, Brevinin 1T, Ranateurin 1, Esculentin 1, Tachyplesin, Androctonin, alpha-defensin, beta-defensin, theta-defensin, defensin (sapecin A), Thionin (crambin), defensin from radish, Drosomycin, Hepcidin, Bac 5, PR-39, Pyrrhocoricin or Histatin 5.

In a further aspect of the present invention at least one of the additional amino acid sequence stretches may be a sushi peptide which is described by Ding J L, Li P, Ho B Cell Mol Life Sci. 2008 April; 65(7-8):1202-19. The Sushi peptides: structural characterization and mode of action against Gram-negative bacteria. Especially preferred is the sushi 1 peptide according to SEQ ID NO: 90. Preferred sushi peptides are sushi peptides 51 and S3 and multiples thereof; FASEB J. 2000 Sep.; 14(12):1801-13.

In a further aspect of the present invention at least one of the additional amino acid sequence stretches is a hydrophobic peptide, which comprises at least 90% of the hydrophobic amino acid residues of valine, isoleucine, leucine, methionine, phenylalanine, tryptophan, cysteine, alanine, tyrosine, proline and/or glycine. In another preferred embodiment the hydrophobic peptide fused to the protein of the invention consists of about 90% to about 95%, or of about 90% to about 100%, or of about 95% to about 100% of the hydrophobic amino acid residues of valine, isoleucine, leucine, methionine, phenylalanine, tryptophan, cysteine, alanine, tyrosine, proline and/or glycine.

Preferred hydrophobic peptides are Walmaghl having the amino acid sequence according to SEQ ID NO: 91 and the hydrophobic peptide having the amino acid sequence Phe-Phe-Val-Ala-Pro (SEQ ID NO: 92).

In a further aspect of the present invention at least one of the additional amino acid sequence stretches is an amphiphatic peptide, which comprises one or more of the positively charged amino acid residues of lysine, arginine and/or histidine, combined to one or more of the hydrophobic amino acid residues of valine, isoleucine, leucine, methionine, phenylalanine, tryptophan, cysteine, alanine, tyrosine, proline and/or glycine. Side chains of the amino acid residues are oriented in order that cationic and hydrophobic surfaces are clustered at opposite sides of the peptide. Preferably, more than about 30, 40, 50, 60 or 70% of the amino acids in said peptide are positively charged amino acids. Preferably, more than about 30, 40, 50, 60 or 70%, of the amino acid residues in said peptide are hydrophobic amino acid residues. Advantageously, the amphiphatic peptide is present at the N-terminal or the C-terminal end of the polypeptide of to the present invention.

In another embodiment of the invention, the amphiphatic peptide consists of at least 5, more preferably at least of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or at least 50 amino acid residues. In a preferred embodiment at least about 30, 40, 50, 60 or 70% of said amino acid residues of the amphiphatic peptide are either arginine or lysine residues and/or at least about 30, 40, 50, 60 or 70% of said amino acid residues of the amphiphatic peptide are of the hydrophobic amino acids valine, isoleucine, leucine, methionine, phenylalanine, tryptophan, cysteine, alanine, tyrosine, proline and/or glycine.

In another preferred embodiment of the present invention the amphiphatic peptide stretch comprises beside the positively charged amino acid residues, in particular lysine and/or arginine residues, hydrophobic amino acid residues, in particular valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues. Preferred are amphiphatic peptide stretches consisting of about 10% to about 50%, or about 20% to about 50%, or about 30% to about 45% or about 5% to about 30% positively charged amino acid residues, in particular lysine and/or arginine residues and of about 50% to about 85%, or about 50% to about 90%, or about 55% to about 90%, or about 60% to about 90%, or about 65% to about 90% hydrophobic amino acid residues, valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues. In another preferred embodiment amphiphatic peptide stretches consisting of 12% to about 50% positively charged amino acid residues, in particular lysine and/or arginine residues and of about 50% to about 85% hydrophobic amino acid residues, valine, isoleucine, leucine, methionine, phenylalanine, tryptophan, cysteine, alanine, tyrosine, proline and glycine residues, more preferably alanine, valine, leucine, isoleucine, phenylalanine, and/or tryptophan residues.

Preferred amphiphatic peptides are a4-helix of T4 lysozyme according to SEQ ID NO: 93 and WLBU2-Variant having the amino acid sequence according to SEQ ID NO: 94 and Walmagh 2 according to SEQ ID NO: 95.

The amino acid sequence stretch may be selected for example from the group of SEQ ID NO: 13-95.

In a preferred embodiment of the present invention the polypeptide of to the present invention comprises two or more amino acid sequence stretches as defined herein. If the polypeptide of to the present invention comprises more than one of these additional amino acid sequence stretches, then it preferably comprises at least two distinct amino acid sequence stretches, preferably selected from the group of amphiphatic peptide, cationic peptide, polycationic peptide, hydrophobic peptide, or naturally occurring antimicrobial peptide, like sushi peptide and defensin. The two or more amino acid sequence stretches, e.g. at the N- or C-terminus of the enzyme and/or at the N- or C-terminus of the polypeptide may thus be two or more distinct cationic peptides or two or more distinct polycationic peptides or two or more distinct antimicrobial peptides or two or more distinct amphiphatic peptides or two or more distinct hydrophobic peptides. The two or more amino acid sequence stretches may in the alternative also be any combination of two or more peptides selected from different representatives of the group consisting of: cationic peptide, a polycationic peptide, a hydrophobic peptide, an antimicrobial peptide, a sushi peptide, a defensin and an amphiphatic peptide. For example, a cationic peptide could be combined with an antimicrobial peptide.

The optional additional amino acid sequence stretches as specified above consist preferably of at least 5, more preferably at least of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 amino acid residues. Especially preferred are those additional amino acid sequence stretches consisting of about 5 to about 100 amino acid residues, about 5 to about 50 or about 5 to about 30 amino acid residues. More preferred are peptide stretches consisting of about 6 to about 42 amino acid residues, about 6 to about 39 amino acid residues, about 6 to about 38 amino acid residues, about 6 to about 31 amino acid residues, about 6 to about 25 amino acid residues, about 6 to about 24 amino acid residues, about 6 to about 22 amino acid residues, about 6 to about 21 amino acid residues, about 6 to about 20 amino acid residues, about 6 to about 19 amino acid residues, about 6 to about 16 amino acid residues, about 6 to about 14 amino acid residues, about 6 to about 12 amino acid residues, about 6 to about 10 amino acid residues or about 6 to about 9 amino acid residues.

In a preferred embodiment the polypeptide of to the present invention comprises at least one, two or more amino acid sequence stretches selected from the group consisting of KRK and SEQ ID NOs: 13-95. Preferably, the polypeptide of to the present invention comprises at least one, two or more amino acid sequence stretches selected from the group consisting of KRK and SEQ ID NOs: 13-95, and an amino acid sequence selected from any one of SEQ ID NOs: 1 to 12, wherein preferably the amino acid sequence stretches, are fused to the N- and/or C-terminus of the amino acid sequence selected from the group consisting of SEQ ID NOs: 1 to 12.

More preferred polypeptides of the invention comprise an amino acid sequence selected from the group consisting of SEQ ID NO: 96-119.

The additional amino acid sequence stretches of the polypeptide comprised in the composition according to the present invention may be linked to the rest of the enzyme by intervening additional amino acid residues e.g. due to cloning reasons. Alternatively, the additional amino acid sequence stretches may be directly linked to the rest of the enzyme sequence without intervening linker sequences. The additional amino acid sequences, if more than one present in the polypeptide of to the present invention and positioned on the same terminus of the enzyme, may likewise be linked to each other by additional intervening amino acid residues or may be directly joined to each other.

Preferably, said intervening additional amino acid residues may not be recognized and/or cleaved by proteases. Preferably said additional amino acid sequences are linked to each other and/or to the enzyme by at least 1, 2, 3, 4, 5, 6, 7, 8, 9 or 10 additional intervening amino acid residues.

In a preferred embodiment the at least one additional amino acid sequence stretch is linked to the rest of the polypeptide of to the present invention, preferably at the N- or C-terminus of the polypeptide of to the present invention, by the additional intervening amino acid residues glycine and serine (Gly-Ser), glycine, serine and serine (Gly-Ser-Ser), glycine, alanine, glycine and alanine (Gly-Ala-Gly-Ala; SEQ ID NO: 120), glycine, alanine, glycine, alanine, glycine, alanine, glycine and alanine (Gly-Ala-Gly-Ala-Gly-Ala-Gly-Ala; SEQ ID NO: 121) or glycine, alanine, glycine, alanine, glycine, alanine, glycine, alanine, glycine, alanine, glycine and alanine (Gly-Ala-Gly-Ala-Gly-Ala-Gly-Ala-Gly-Ala-Gly-Ala; SEQ ID NO: 122).

Aside of the peptidoglycan hydrolase and the optional additional amino acid sequence stretches, as defined herein, the polypeptide of to the present invention may of course also comprise other amino acid sequence elements, e.g. one or more tags, e.g. a His-tag, Strep-tag, Avi-tag, Myc-tag, Gst-tag, JS-tag, cystein-tag, FLAG-tag or other tags known in the art, thioredoxin, maltose binding proteins (MBP) etc.

In this context, the polypeptide of to the present invention, preferably having the ability of degrading the peptidoglycan layer, may additional comprise a tag e.g. for purification.

Preferred is a His₆-tag, preferably at the C-terminus and/or the N-terminus of the polypeptide of to the present invention. Said tag can be linked to the polypeptide by additional amino acid residues e.g. due to cloning reasons. Preferably said tag can be linked to the protein by at least 1, 2, 3, 4, 5, 6, 7, 8, 9 or 10 additional amino acid residues. Preferably said additional amino acid residues may not be recognized and/or cleaved by proteases. In a preferred embodiment the polypeptide of to the present invention comprises a His₆-tag at its C-terminus linked to the polypeptide by the additional amino acid residues lysine and glycine (Lys-Gly) or leucine and glutamic acid (Leu-Glu). Preferably, said additional amino acid residues may be not recognized or cleaved by proteases. In another preferred embodiment the polypeptide of to the present invention comprises a His₆-tag at its N-terminus linked to the polypeptide by the additional amino acid residues lysine and glycine (Lys-Gly) or leucine and glutamic acid (Leu-Glu). In another preferred embodiment the polypeptide comprises a His₆-tag at its N-and C-terminus linked to the polypeptide by the additional amino acid residues lysine and glycine (Lys-Gly) or leucine and glutamic acid (Leu-Glu). A preferred polypeptide of the invention is listed in SEQ ID NO: 123.

The polypeptide and/or the pharmaceutical composition of the present invention may be administered to a subject in an effective amount, wherein the subject may be a human being or an animal, preferably a livestock or companion animal. In particular, the polypeptide and/or the pharmaceutical composition of the present invention may be used for the treatment of chronic bacterial infections caused by Gram-negative and/or Gram-positive bacteria. The treatment may comprise the treatment of infections of the skin, of soft tissues, the respiratory system, the lung, the digestive tract, the eye, the ear, the teeth, the nasopharynx, the mouth, the bones, the vagina, of wounds of bacteraemia and/or endocarditis, e.g. caused by Gram-negative and/or Gram-positive bacteria, in particular by the bacteria as mentioned herein.

The dosage and route of administration used for the treatment according to the present invention depends on the specific disease and/or site of infection to be treated. The route of administration may be for example oral, topical, nasopharyngeal, parenteral, intravenous, rectal or any other route of administration.

For application of the polypeptide and/or the pharmaceutical composition of the present invention to a site of infection a formulation may be used that protects the active compounds from environmental influences such as proteases, oxidation, immune response etc., until it reaches the site of infection. Therefore, the formulation may be capsule, dragee, pill, powder, suppository, emulsion, suspension, gel, lotion, cream, salve, injectable solution, syrup, spray, inhalant or any other medical reasonable galenic formulation. Preferably, the galenic formulation may comprise suitable carriers, stabilizers, flavourings, buffers or other suitable reagents. For example, for topical application the formulation may be a lotion, cream, gel, salve or plaster, for nasopharyngeal application the formulation may be saline solution to be applied via a spray to the nose. For oral administration in case of the treatment of a specific infection site e.g. in the intestine, it can be necessary to protect polypeptide of to the present invention from the harsh digestive environment of the gastrointestinal tract until the site of infection is reached.

In one aspect of the present invention the subject suffering from a chronic bacterial infection has been treated with antibiotics whereby the symptoms of the bacterial infection has been reduced or cured but the bacterial infection reoccurred after offsetting the antibiotics.

Preferably, the polypeptide and/or the pharmaceutical composition of the present invention is used for the treatment of chronic bacterial infections, wherein the infection is caused by multiresistant or antibiotics tolerant bacterial strains, in particular by strains resistant or tolerant against one or more antibiotics selected from the following group: beta-lactams, aminoglycosides, fluoroquinolones, macrolides, novobiocin, rifampicin, oxazolidinones, fusidic acid, mupirocin, pleuromutilins, daptomycin, vancomycin, tetracyclines, sulfonamides, chloramphenicol, trimetoprim, fosfomycin, cycloserine, polymyxin, streptomycin, tetracycline, cephalothin, gentamicin, cefotaxime, cephalosporin, ceftazidime or imipenem. Furthermore, the polypeptide and/or the pharmaceutical composition of the present invention can be used for treatment by administering in combination with conventional antibacterial agents, such as antibiotics.

The present invention also relates to a pharmaceutical pack comprising one or more compartments, wherein at least one compartment comprises the polypeptide and/or the pharmaceutical composition of the present invention.

In another aspect the present invention relates to a process of preparation of a pharmaceutical composition, said process comprising admixing one or more of the polypeptide and/or the pharmaceutical composition of the present invention with a pharmaceutically acceptable diluent, excipient or carrier.

Preferably the polypeptide and/or the pharmaceutical composition of the present invention is used as a medicament for the treatment of a chronic bacterial infection caused by Enterobacteriaceae, in particular Escherichia, Salmonella, Shigella, Citrobacter, Edwardsiella, Enterobacter, Hafnia, Klebsiella, Morganella, Proteus, Providencia, Serratia, and Yersinia; Pseudomonadaceae, in particular Burkholderia, Pseudomonas, Stenotrophomonas, Shewanella, Sphingomonas and Comamonas; Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Spirochaetaceae, in particular Treponema and Borrelia; Leptospiraceae, Campylobacter, Helicobacter, Spirillum, Streptobacillus, Bacteroidaceae, in particular Bacteroides, Fusobacterium, Prevotella and Porphyromonas; Acinetobacter, in particular A. baumanii; Listeria monocytogenes, Staphylococcus aureus, Enterococcus faecalis, Enterococcus faecium, Streptococcus pneumoniae, Streptococcus pyogenes, Streptococcus uberis, Streptococcus agalacticae, Streptococcus mutans, Streptococcus equi, Clostridium difficile, Clostridium botulinum, Clostridium tetani, Clostridium perfringens, Bacillus anthracis, Bacillus cereus, Propionibacterium acnes, Mycobacterium avium, Mycobacterium abscessus, Mycobacterium tuberculosis, Corynebacterium diphteriae, Mycoplasma pneumonia and Actinomyces.

In a specific embodiment of the present invention the polypeptide and/or the pharmaceutical composition of the present invention is used as a medicament for the treatment of a chronic bacterial infection caused by Listeria monocytogenes, in particular Granulomatosis infantiseptica (listeriosis of newborns), mononucleosis, conjunctivitis, meningitis, granulomatosis septica and the listeriosis of pregnant women.

In another specific embodiment of the present invention the polypeptide and/or the pharmaceutical composition of the present invention is used in the manufacture of a medicament for the treatment of a chronic bacterial infection caused by Staphylococcus aureus, in particular infections of the skin like pyoderma, particularly folliculitis, furuncle, carbuncle, abscesses of the sweat glands and pemphigus, and like scaled skin syndrome. The scaled skin syndrome can appear in three clinical pictures: dermatitis exfoliativa, impetigo bullosa and scarlatiniform erythroderma. Moreover chronic bacterial infection caused by Staphylococcus aureus are Staphylococcus pneumonia, hospitalism, in particular surgical wound infections, mastitis puerperalis and enterokolitis, and food poisonings.

In another specific embodiment of the present invention the polypeptide and/or the pharmaceutical composition of the present invention is used in the manufacture of a medicament for the treatment of a chronic bacterial infection caused by Streptococcus pyogenes, in particular tonsillitis, pharyngitis, scarlet, erysipelas, rheumatic fever and acute glomerulonephritis.

In another specific embodiment of the present invention the polypeptide and/or the pharmaceutical composition of the present invention is used in the manufacture of a medicament for the treatment of a chronic bacterial infection caused by Streptococcus pneumoniae, in particular pneumonia, ulcus serpens corneae, otitis media, meningitis, peritonitis, mastoiditis and osteomyelitis.

In another specific embodiment of the present invention the polypeptide and/or the pharmaceutical composition of the present invention is used in the manufacture of a medicament for the treatment of a chronic bacterial infection caused by Clostridium perfringens, in particular gas gangrene, enteritis necroticans ulcerosa and food poisonings.

In another specific embodiment of the present invention the polypeptide and/or the pharmaceutical composition of the present invention is used in the manufacture of a medicament for the treatment of a chronic bacterial infection caused by Clostridium botulinum, in particular botulism.

In another specific embodiment of the present invention the polypeptide and/or the pharmaceutical composition of the present invention is used in the manufacture of a medicament for the treatment of a chronic bacterial infection caused by Clostridium difficile, in particular pseudomembranous colitis (or enterocolitis).

In another specific embodiment of the present invention the polypeptide and/or the pharmaceutical composition of the present invention is used in the manufacture of a medicament for the treatment of a chronic bacterial infection caused by Bacillus anthracis, in particular cutaneous anthrax, inhalation anthrax, and gastrointestinal anthrax.

In another specific embodiment of the present invention the polypeptide and/or the pharmaceutical composition of the present invention is used in the manufacture of a medicament for the treatment of a chronic bacterial infection caused by Enterococcus faecalis or E. faecium, like nosokomial infections, and endokarditis.

In another specific embodiment of the present invention the polypeptide and/or the pharmaceutical composition of the present invention is used in the manufacture of a medicament for the treatment of a chronic bacterial infection caused by Bacillus cereus, in particular food poisonings, bronchial pneumonia, septicaemia and meningitis.

In another specific embodiment of the present invention the polypeptide and/or the pharmaceutical composition of the present invention is used in the manufacture of a medicament for the treatment of a chronic bacterial infection caused by Mycobacterium avium, Mycobacterium paratuberculosis, Mycobacterium abscessus and Mycobacterium tuberculosis, in particular tuberculosis.

In another specific embodiment of the present invention the polypeptide and/or the pharmaceutical composition of the present invention is used in the manufacture of a medicament for the treatment of a chronic bacterial infection caused by Mycoplasma pneumoniae, in particular pneumonia, diseases of the upper respiratory tract and inflammations of the ear drum.

In another specific embodiment of the present invention the polypeptide and/or the pharmaceutical composition of the present invention is used in the manufacture of a medicament for the treatment of a chronic bacterial infection caused by Actinomyces, in particular actinomycosis in human, cattle, cat and dog.

In another specific embodiment of the present invention the polypeptide and/or the pharmaceutical composition of the present invention is used in the manufacture of a medicament for the treatment of a chronic bacterial infection caused by Corynebacterium diphteriae, in particular localized diphtheria of the tonsils, the nose, the nasopharynx or the middle ear, progressive diphtheria of the larynx, the trachea and the bronchi, toxic or maligne diphtheria, skin and wound diphtheria.

In another specific embodiment of the present invention the polypeptide and/or the pharmaceutical composition of the present invention is used in the manufacture of a medicament for the treatment of a chronic bacterial infection caused by E. coli , in particular for urinary tract infections, diseases of the digestive tract like Morbus Crohn and Colitis ulcerosa.

In another specific embodiment of the present invention the polypeptide and/or the pharmaceutical composition of the present invention is used in the manufacture of a medicament for the treatment of a chronic bacterial infection caused by Pseudomonas, in particular urinary tract infections, respiratory system infections, dermatitis, soft tissue infections, bacteraemia and a variety of systemic infections, particularly in victims of severe burns, and also in cancer and AIDS patients who are immuno-compromised.

In another specific embodiment of the present invention the polypeptide and/or the pharmaceutical composition of the present invention is used in the manufacture of a medicament for the treatment of a chronic bacterial infection caused by Acinetobacter, e.g. Acinetobacter baumanii, in particular wound infections, lung infections and meningitis (often also caused by A. lwoffii).

A further aspect is a method of treating chronic bacterial infections by applying a polypeptide comprising a peptidoglycan hydrolase or a pharmaceutical composition comprising a polypeptide comprising a peptidoglycan hydrolase to a human or animal.

The polypeptide of to the present invention can be produced by standard means known in the art, e.g. by recombinant expression of nucleic acids encoding the respective polypeptide in appropriate host cells. If the polypeptide of to the present invention comprises for example addtionally amino acid sequence stretches or tags, such fusion proteins may be produced by linking the required individual nucleic acid sequences using standard cloning techniques as described e.g. by Sambrook et al. 2001, Molecular Cloning: A Laboratory Manual. Such a polypeptide may be produced likewise with methods known in the art, e.g., in recombinant DNA expression systems.

It has to be noted that the polypeptide of to the present invention having peptidoglycan hydrolase activity on thus the activity of degrading the peptidoglycan layer of bacteria can be assembled like using a tool box, i.e. any additional amino acid sequence stretch and antimicrobial peptide disclosed above may be included in the polypeptide of to the present invention. Consequently, it is possible to construct a suitable polypeptide for any bacteria which should be eliminated.

It should be understood that the detailed description and specific examples disclosed herein, indicating particular embodiments of the invention, are given by way of illustration only, since various changes and modifications within the spirit and scope of the invention will become apparent to those skilled in the art from this description.

### EXAMPLE 1

Persister killing—Planktonic persister cells were isolated from a stationary phase culture as described e.g. in Chen et al., 2011(Chen X, Zhang M, Zhou C, Kallenbach N R, Ren D. 2011. Control of bacterial persister cells by Trp/Arg-containing antimicrobial peptides. Appl Environ Microbiol 77:4878-4885.). Briefly, an overnight culture of P. aeruginosa PA14 and PA1255 (a cystic fibrosis isolate) was inoculated each in 50 ml 1:20 Trypticase Soy Broth (TSB). After 48 h of growth at 37° C., the culture was treated with ofloxacin at a final concentration of 10 μg/ml (5× MIC) for 5 hours at 37° C., while shaking at 200 rpm. The persister cells surviving this antibiotic treatment were isolated by centrifugation (5250 g, 15 min, 4° C.) and the cell pellet was washed twice with 20 mM HEPES buffer. Prior to treatment, cells were resuspended in the same volume of 20 mM HEPES buffer. A persister killing assay was performed as described e.g. by De Groote et al., 2009 (De Groote V N, Verstraeten N, Fauvart M, Kint C I, Verbeeck A M, Beullens S, Cornelis P, Michiels J. 2009. Novel persistence genes in Pseudomonas aeruginosa identified by high-throughput screening. FEMS Microbiol Lett 297:73-79). Briefly, a volume of 100 μl of the isolated persister fraction was mixed with polypeptides comprising peptidoglycan hydrolase or ciprofloxacin (final concentrations of 10× or 30× MIC against the respective strains), in the absence or presence of 0.5 mM EDTA-Na2 (final concentration) up to a final volume of 200 μl. Control treatments with 20 mM HEPES buffer, 0.5 mM EDTA or ofloxacin (30×and 10× MIC) were performed in parallel. The mixtures were shaken (200 rpm) for lh at 37° C. Treated persisters where washed twice with 20 mM HEPES buffer and appropriate dilutions were plated on TSB agar plates and incubated at 37° C. The number of persisters was determined after 72 h incubation to allow complete resuscitation of all surviving persisters. Each experiment was independently repeated at least 3 times.

### EXAMPLE 2

### Materials and Methods for Examples 3 to 6

Isolation of Persister Fraction

Planktonic persister cells were isolated from a stationary-phase culture as described previously (Liebens V, Defraine V, Van der Leyden A, De Groote V N, Fierro C, Beullens S, Verstraeten N, Kint C, Jans A, Frangipani E, Visa P, Marchal K, Versées W, Fauvart M, Michiels J. 2014. A putative de-N-acetylase of the PIG-L superfamily affects fluoroquinolone tolerance in Pseudomonas aeruginosa. Pathogen. Dis. 71:39-54.) with minor modifications. An overnight culture of P. aeruginosa PA14, PAl255 (a cystic fibrosis isolate), Escherichia coil UTI-89 (isolate from chronic urinary tract infection) and Acinetobacter baumannii RUH134 (reference strain for global clone 2) was diluted 100×in 50 ml 1:20 Trypticase soy broth (TSB). After overnight growth at 37° C., the cultures were treated with either 10 μg/ml ofloxacin (P. aeruginosa strains), 200 μg/ml tobramycin (A. baumannii) or 200 μg/ml amikacin (E. coli) for 5 h at 37° C., while shaking at 200 rpm. The antibiotic concentrations were chosen to allow only persister cells to survive, based on previous time-kill curves. The persister fractions were isolated by centrifugation (5,250× g for 15 min at 4° C.), and the cell pellet was washed twice with 20 mM HEPES (pH 7.4). Prior to treatment, the cells were resuspended in a ten-fold reduced volume of 20 mM HEPES (pH 7.4) to concentrate the persisters.

Persister Killing Assay

A persister killing assay was performed as described previously (Liebens et al., 2014), with minor modifications. A volume of 100 μl of the isolated persister fraction was mixed with either the polypeptide according to SEQ ID NO: 124, 125, 126 or ciprofloxacin (final concentrations of 10× or 30× MIC against the respective strains), in the absence or presence of 0.5 mM EDTA-Na₂ (final concentration) up to a final volume of 200 μl. Control treatments with 20 mM HEPES (pH 7.4), 0.5 mM EDTA, or ofloxacin/tobramycin/amikacin (30× and 10× MIC) were performed in parallel. The mixtures were shaken (200 rpm) for lh at 37° C. The treated persister cells were washed twice with 20 mM HEPES (pH 7.4), and the appropriate dilutions were plated on TSB plates and incubated at 37° C. The number of persisters was determined after 72 h of incubation to allow for complete resuscitation of all surviving persisters.

### EXAMPLE 3

The bactericidal activity of the polypeptide according to SEQ ID NO: 126 was tested against isolated persister fractions of P. aeruginosa strains. Persister fractions of two different strains (PA14 and PAl255, an isolate of a cystic fibrosis patient) were collected after 5 h exposure to 5× MIC ofloxacin. The lack of killing after further exposure of the isolated fractions to increased ofloxacin concentrations (10× and 30× MIC) confirmed the persistent (antibiotic-tolerant) nature of both isolated persister fractions. The persisters were incubated with different concentrations of the polypeptide according to SEQ ID NO: 126 or ciprofloxacin (10× MIC and 30× MIC) in the absence or presence of 0.5 mM EDTA. The polypeptide according to SEQ ID NO: 126 (10× and 30× MIC) completely eradicated the remaining persisters in the presence of 0.5 mM EDTA, whereas similar doses of ciprofloxacin (with and without EDTA) or 0.5 mM EDTA alone did not affect the persisters. In the absence of EDTA, a 30× MIC dose of the polypeptide according to SEQ ID NO: 126 reduced the persister fractions of PA14 and PAl255 severely. In summary, the overall activity of the polypeptide according to SEQ ID NO: 126 against these persister fractions is extremely pronounced.

### EXAMPLE 4

The killing activity of the polypeptide according to SEQ ID NO: 126 (30× MIC) was evaluated against A. baumannii RUH persisters. Those were isolated after a 5 h treatment with tobramycin. Aside of the polypeptide according to SEQ ID NO: 126, also the effect of the polypeptide according to SEQ ID NO: 125 (30× MIC) was evaluated. Ciprofloxacin (30× MIC) was used as a control antibiotic. Both polypeptides, i.e. the polypeptide according to SEQ ID NO: 126 as well as the polypeptide according to SEQ ID NO: 125, and ciprofloxacin were tested in the absence and presence of 0.5 mM EDTA. Tobramycin (30× MIC) and 0.5 mM EDTA were added as a control. The polypeptide according to SEQ ID NO: 126 (30× MIC) reduced the number of persisters with approximately 1.75 log units, whereas the polypeptide according to SEQ ID NO: 126 (30× MIC) with 0.5 mM EDTA resulted in a complete eradication (>2.5 log in comparison to 0.5 mM EDTA/Tobramycin control). Whereas the polypeptide according to SEQ ID NO: 125 (30× MIC) alone did not strongly affect persisters, a strong reduction (approximately 1.8 log in comparison to the 0.5 mM EDTA/Tobramycin control) is observed in the presence of 0.5 mM EDTA.

### EXAMPLE 5

The killing effect of the polypeptide according to SEQ ID NO: 124 (comprising a fusion of Sarcotoxin IA (SEQ ID NO: 48) and KZ144 (SEQ ID NO: 11), active against vegetative cells of E. coli) was analyzed against the isolated persister fraction of E. coli. An isolate of a chronic urinary tract infection was used to isolate the persister fraction upon 5 h amikacin exposure. A final concentration corresponding to 10× MIC was used, both in the absence or the presence of 0.5 mM EDTA. 0.5 mM EDTA was included as control. Ciprofloxacin (10× MIC) was tested as comparison antibiotic. The isolated persister fraction (approximately 10² persisters/ml) was significantly smaller in comparison to P.aeruginosa and A. baumannii persister fractions. The control antibiotic ciprofloxacin (10× MIC) (with and without EDTA) and the polypeptide according to SEQ ID NO: 124 (10× MIC) without 0.5 mM EDTA did not completely kill persisters. However, combination of the polypeptide according to SEQ ID NO: 124 (10× MIC) with 0.5 mM EDTA completely eradicated the persisters.

### EXAMPLE 6

The anti-persister effect of the polypeptide according to SEQ ID NO: 124 was evaluated against persisters isolated from P. aeruginosa PA14 upon exposure to ofloxacin. The persisters were treated with the polypeptide according to SEQ ID NO: 124 with a final concentration equivalent to 30× MIC against P. aeruginosa PA14, both in the absence and the presence of 0.5 mM EDTA. 0.5 mM EDTA and ofloxacin (30× MIC) were included as a control. Ciprofloxacin was included as comparison antibiotic. In comparison to the 0.5 mM EDTA control, the polypeptide according to SEQ ID NO: 124 (30× MIC) reduced the number of persisters with approximately 2.3 log units. When 0.5 mM EDTA is added to the polypeptide according to SEQ ID NO: 124 (30× MIC), a complete eradication is achieved (approximately 5 log units).

1. A method of treating a chronic bacterial infection in a subject comprising administering to said subject an endolysin, wherein the chronic bacterial infection is associated with Pseudomonas, Escherichia, Staphylococcus aureus, Acinetobacter, Mycobacterium avium, Mycobacterium tuberculosis, Listeria monocytogenes and/or Salmonella bacteria.

2. The method of claim 1, wherein the endolysin is an endopeptidase, N-acetyl-muramoyl-L-alanine-amidase, N-acetyl-muramidase, N-acetyl-glucosaminidase and/or lytic transglycosylase.

3. The method of claim 1, wherein the endolysin is PhiV10p30 of phage ΦV10 STM0907.Fels0 of phage FELS-1, epsilon15p25 of phage ε15, YuA20 of phage YUA, ORF23 of phage B3, BcepMu22 of phage BcepMu, F116p62 of phage F116, STM2715.S.Fels2 of phage Fels2, gp76 of phage ES18, SPSV3_gp23 of phage SETP3, phi32_17 of phage ΦECO32, HK022p54 of phage HK022, HK97p58 of phage HK97, HK620p36 of phage HK620, VIP0007 of phage E1, Sf6p62 of phage SF6, R (SfVp40) of phage SFV, gp22 of phage BCEPC6B, K (P2p09) of phage P2, K (Wphi09) of phage WΦ, rv5_gp085 of phage RV5, EpJS98_gp116 of phage JS98, gp3.5 of phage 13A, gp3.5 of phage BA14, gp3.5 of phage ECODS1, CKV1F_gp16 of phage K1F, T3p18 of phage T3, gh-1p12 of phage GH-1, gp3.5 of phage K11, ORF12 of phage ΦCTX, Bcep43-27 of phage BCEP43, Bcep781-27 of phage BCEP781, Bcep1-28 of phage BCEP1, BcepNY3gene26 of phage BCEPNY3, gp45 of phage ΦE12-2, gp28 of phage Φ52237, P27p30 of phage ΦP27, RB49p102 of phage RB49, phi1-p102 of phage Φ1, lys (T5.040) of phage T5, YP_001956952.1 of phage 201phi2-1, Aeh1p339 of phage Aehl, YYZgp45 of phage YYZ-2008, gp144 of phage ΦKZ, EL188 of phage EL, YP_001671940.1 of the phage LUZ24, N4 N-acetylmuramidase of phage N4gp61, STM0016 endolysin, PSP3 endolysin, PVP-SE1gp146 of phage PVPSE1, PlyA118, PlyA500, PlyPSA, PlyA511, PlyP35, PlyP40, Phi 11, Phi MR11, LysK, PlyS9, Ply3626, CD27L, B30, phage Dp-1 encoded Pal amidase, C1endolysin PlyC, Cpl-1 endolysin, PlyGBS, PlyV12, Phage gamma endolysin PlyG or an endolysin having an amino acid sequence selected from the group of SEQ ID NO: 1-11.

4. The method claim 1, wherein said endolysin further comprises an non-endolysin amino acid sequence segment.

5. The method of claim 4, wherein the amino acid segment is LL-37, SMAP-29, Indolicidin, Protegrin, Cecropin P1, Magainin, Pleurocidin, Cecropin A (A.aegypti), Cecropin A (D. melanogaster), Buforin II, Sarcotoxin IA, Apidaecin, Ascaphine 5, Nigrocine 2, Pseudin 1, Ranalexin, Melittin, Lycotoxin 1, Parasin 1, Buforin I, Dermaseptin 1, Bactenecin 1, Thanatin, Brevinin 1T, Ranateurin 1, Esculentin 1, Tachyplesin, Androctonin, alpha-defensin, beta-defensin, theta-defensin, defensin (sapecin A), Thionin (crambin), defensin from radish, Drosomycin, Hepcidin, Bac 5, PR-39, Pyrrhocoricin or Histatin 5.

6. The method of claim 4, wherein the amino acid segment comprises an amino acid sequence selected from the group of SEQ ID NO: 13-95.

7. The method of claim 1, wherein the bacterial infection is an infection of the skin, of soft tissues, the respiratory system, the lung, the digestive tract, the eye, the ear, the teeth, the nasopharynx, the mouth, the bones, and/or the vagina.

8. The method of claim 1, wherein the chronic bacterial infection is associated with Pseudomonas aeruginosa, Escherichia coli and/or Acinetobacter baumannii bacteria.

9. The method of claim 1, wherein the chronic bacterial infection is associated with Pseudomonas aeruginosa.

10. The method of claim 1, wherein the chronic bacterial infection is associated with Escherichia coli.

11. The method of claim 1, wherein the chronic bacterial infection is associated with Acinetobacter baumannii.

12. A pharmaceutical composition comprising an endolvsin formulated for the treatment of chronic bacterial infections, wherein the chronic bacterial infection is associated with Pseudomonas, Escherichia, Staphylococcus aureus, Acinetobacter, Mycobacterium avium, Mycobacterium tuberculosis, Listeria monocytogenes and/or Salmonella bacteria.

13. The pharmaceutical composition of claim 12, wherein the chronic bacterial infection is associated with Pseudomonas aeruginosa, Escherichia coli and/or Acinetobacter baumannii bacteria.

14. The pharmaceutical composition of claim 12, wherein the bacterial infection is an infections of the skin, of soft tissues, the respiratory system, the lung, the digestive tract, the eye, the ear, the teeth, the nasopharynx, the mouth, the bones, and/or the vagina.
